SEQUENCE LISTING (11 sequences)

SEQ ID NO: 1 - protein, 121 aa, synthetic construct
  REGION 1..121: note = Synthetic Vh Omalizumab
EVQLVESGGGLVQPGGSLRLSCAVSGYSITSGYSWNWIRQAPGKGLEWVASITYDGSTNYADSVKGRFTISRDDSKNTFYLQMNSLRAEDTAVYYCARGSHYFGHWHFAVWGQGTLVTVSS

SEQ ID NO: 2 - protein, 111 aa, synthetic construct [sequence omitted]

SEQ ID NO: 3 - protein, 447 aa, synthetic construct
  REGION 1..447: note = Synthetic WT heavy chain anti-IgE
EVQLVESGGGLVQPGGSLRLSCAVSGYSITSGYSWNWIRQAPGKGLEWVASITYDGSTNYADSVKGRFTISRDDSKNTFYLQMNSLRAEDTAVYYCARGSHYFGHWHFAVWGQGTLVTVSSGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 447 aa, synthetic construct
  REGION 1..447: note = Synthetic Fc LALA
EVQLVESGGGLVQPGGSLRLSCAVSGYSITSGYSWNWIRQAPGKGLEWVASITYDGSTNYADSVKGRFTISRDDSKNTFYLQMNSLRAEDTAVYYCARGSHYFGHWHFAVWGQGTLVTVSSGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 446 aa, synthetic construct
  REGION 1..446: note = Synthetic Fc NA
VQLVESGGGLVQPGGSLRLSCAVSGYSITSGYSWNWIRQAPGKGLEWVASITYDGSTNYADSVKGRFTISRDDSKNTFYLQMNSLRAEDTAVYYCARGSHYFGHWHFAVWGQGTLVTVSSGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 215 aa, synthetic construct
  REGION 1..215: note = Synthetic Light chain common to the WT, LALA and NA anti-IgE mAbs
DIQLTQSPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 7 - protein, 324 aa, synthetic construct
  REGION 1..324: note = Synthetic Wild-type human IgE-Fc (CE2-CE4 domains with numbering V224-K547according to Dorrington & Bennich (1978)
VASRDFTPPTVKILQSSCDGGGHFPPTIQLLCLVSGYTPGINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 8 - protein, 453 aa, synthetic construct
  REGION 1..453: note = Synthetic Heavy chain sequence of the Ligelizumab
QVQLVQSGAEVMKPGSSVKVSCKASGYTFSWYWLEWVRQAPGHGLEWMGEIDPGTFTTNYNEKFKARVTFTADTSTSTAYMELSSLRSEDTAVYYCARFSHFSGSNYDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 214 aa, synthetic construct
  REGION 1..214: note = Synthetic Light chain sequence of the Ligelizumab
EIVMTQSPATLSVSPGERATLSCRASQSIGTNIHWYQQKPGQAPRLLIYYASESISGIPARFSGSGSGTEFTLTISSLQSEDFAVYYCQQSWSWPTTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 447 aa, synthetic construct
  REGION 1..447: note = Synthetic Heavy chain sequence of the Quilizumab
EVQLVESGGGLVQPGGSLRLSCAASGFTFSDYGIAWVRQAPGKGLEWVAFISDLAYTIYYADTVTGRFTISRDNSKNTLYLQMNSLRAEDTAVYYCARDNWDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 219 aa, synthetic construct
  REGION 1..219: note = Synthetic Light chain sequence of the Quilizumab
DIQMTQSPSSLSASVGDRVTITCRSSQSLVHNNANTYLHWYQQKPGKAPKLLIYKVSNRFSGVPSRFSGSGSGTDFTLTISSLQPEDFATYYCSQNTLVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0076410 A1
(43) Pub. Date: Mar. 7, 2024

(54) FC-ENGINEERED ANTI-HUMAN IGE ANTIBODIES AND METHODS OF USE

(71) Applicants: INSERM (INSTITUT NATIONAL DE LA SANTÉ ET DE LA RECHERCHE MÉDICALE), Paris (FR); INSTITUT PASTEUR, Paris (FR)

(72) Inventors: Laurent REBER, Paris (FR); Pierre BRUHNS, Paris (FR); Bianca BALBINO, Paris (FR)

(21) Appl. No.: 18/312,921

(22) Filed: May 5, 2023

Related U.S. Application Data
(62) Division of application No. 17/046,838, filed on Oct. 12, 2020, now abandoned.
(60) Provisional application No. 62/657,401, filed on Apr. 13, 2018.

(30) Foreign Application Priority Data
Mar. 29, 2019 (EP) 19305418.6

(51) Int. Cl. C07K 16/42; A61P 37/06

(52) U.S. Cl. CPC C07K 16/4291; A61P 37/06; A61K 2039/505

(57) ABSTRACT

The present invention relates to the treatment of IgE-mediated disease. The inventors hypothesized that formation of immune complexes between Omalizumab and IgE might be responsible for some of the adverse reactions observed in highly atopic patients (i.e. patients with a history of anaphylaxis and/or high IgE titers). Immune complexes can induce inflammation through activation of Fc gamma receptors (FcγRs) and/or the complement cascade. They identified that Omalizumab:hIgE immune complexes activate human FcγRs in vitro. Moreover, similarly to some of the reported side effects observed in human, Omalizumab:hIgE immune complexes can induce anaphylaxis when injected in mice expressing human FcγRs. Using publicly available omalizumab VH and VL sequences, they cloned and produced two mutant versions of omalizumab in which residues in the Fc portion of the Ab were mutated. These variants did not induce anaphylaxis when injected into mice expressing human FcγRs and could be thus used for the treatment of IgE-mediated disease. Thus invention relates to a recombinant immunoglobulin heavy chain protein which comprises at least one mutation in the Fc portion and recombinant antibody comprising said heavy chain protein.

### SEQUENCE LISTING

This document incorporates by reference an electronic sequence listing file, which was electronically submitted along with this document. The xml file is named 2023-05-05_11450648US2_seqlisting.xml, is 18,429 bytes, and was created on May 4, 2023.

### FIELD OF THE INVENTION

The present invention relates a recombinant immunoglobulin heavy chain protein which comprises at least one mutation in the Fc portion.

### BACKGROUND OF THE INVENTION

IgE antibodies (Abs) are key mediators of allergic diseases. Quantification of allergen-specific IgE is the main test used in clinic for the diagnosis of allergy (Hamilton & Adkinson 2004 J Allergy Clin Immun 114, 213-225). Upon exposure to an allergen in allergic patients, such allergen is recognized by IgE bound to their high affinity receptor FcεRI on the surface of mast cells, basophils and other effector cells, which promotes the immediate activation of these cells, and the release of both preformed and newly synthesized inflammatory mediators such as histamine, responsible for allergic symptoms (Galli & Tsai, Nat Med. 2012 18(5):693-704).

Omalizumab is a recombinant humanized IgG1 anti-human IgE mAb produced jointly by Novartis and Genentech under the trade name Xolair. Omalizumab is approved for the treatment of severe asthma (Busse et al., J Allergy Clin Immunol. 2001 August; 108(2):184-90) and chronic spontaneous urticaria (Maurer et al. 2013, N Engl J Med. 368(10):924-35), and shows promises for the treatment of other allergic diseases, including food allergy (MacGinnitie et al., J Allergy Clin Immunol. 2017 139(3):873-881.e8). Omalizumab inhibits the binding of human IgE (hIgE) Abs to their high affinity receptor FcεRI. Reduction of hIgE on the surface of FcεRI-bearing mast cells and other effector cells limits the degree of mediators release upon allergen contact, including histamine (Kaplan et al., Allergy. 2017 April; 72(4):519-533). In addition, long-term treatment with Omalizumab also reduces the numbers of FcεRI receptors on the surface of mast cells, basophils and other effector cells in allergic patients (Kaplan et al., Allergy. 2017 April; 72(4):519-533).

Treatment with Omalizumab is associated with adverse reactions, ranging from skin inflammation (45% of reported side effects), to severe systemic allergic shock (i.e. ‘anaphylaxis’) in −0.2% of patients (Limb et al., J Allergy Clin Immunol, 2007 120:1378-1381; Lieberman et al., J Allergy Clin Immunol, 2016 138(3):913-915.e2). In 2000, the US Food and Drug Administration requested the suspension of new Omalizumab trials because the development of thrombocytopenia was reported in monkey studies (BioDrugs. 2002; 16(5):380-6). As a result, the FDA has now restricted the use of Omalizumab to patients with severe asthma and serum IgE levels below 700 IU/mL (1.68 μg/ml), therefore precluding its use in patients with high IgE titers. Accordingly, there is a long felt and unmet need for anti-human IgE antibody alternatives to Omalizumab that are safer and effective.

### SUMMARY OF THE INVENTION

Omalizumab-induced severe adverse reactions are more frequent in patients with a history of anaphylaxis (Lieberman et al., J Allergy Clin Immunol, 2016 138(3):913-915.e2). The inventors thus hypothesized that formation of immune complexes between Omalizumab and IgE might be responsible for some of the adverse reactions observed in highly atopic patients (i.e. patients with a history of anaphylaxis and/or high IgE titers). Immune complexes can induce inflammation through activation of Fc gamma receptors (FcγRs) and/or the complement cascade. They identified that Omalizumab:hIgE immune complexes activate human FcγRs in vitro. Moreover, similarly to some of the reported side effects observed in human, Omalizumab:hIgE immune complexes can induce anaphylaxis when injected in mice expressing human FcγRs. They thus tested this novel hypothesis.

Using publicly available omalizumab VH and VL sequences (www.drugbank.ca/drugs/DB00043), they cloned and produced two mutant versions of omalizumab in which residues in the Fc portion of the Ab were mutated (L234A/L235A, or N297A anti-hIgE mAbs). They hypothesized this would reduce binding to FcγRs and complement (Scields et al., J Biol Chem. 2001 276(9):6591-604; Hezareh et al., J Virol 2001; 75:12161-12168; Tao et al., J Immunol 1989; 143:2595-2601). They found that Omalizumab and the L234A/L235A or N297A mutant anti-hIgE mAbs were equally capable of recognizing and blocking human IgE (hIgE) in vitro and in vivo in mice humanized for the IgE receptor FcεRI. However, immune complexes made of hIgE and L234A/L235A or N297A mutant anti-hIgE mAbs had markedly reduced binding to FcγRs as compared to immune complexes made of hIgE and omalizumab, and did not induce anaphylaxis when injected into mice expressing human FcγRs.

Their results show that in certain embodiments L234A/L235A or N297A mutant anti-hIgE mAbs represent novel and safer therapeutic solution for the treatment of allergic reactions, with reduced FcγR- and complement-dependent adverse reactions as compared to Omalizumab. More, these results proved that any mutations on Fc portion inhibiting or reducing the binding of the immune complexe made of IgE and mutated IgG to FcγRs in all anti-IgE antibodies, will reduce or abolsih the adverse reactions and especially anaphylaxis of these anti-IgE.

Accordingly, the sequences of the recombinant immunoglobulin heavy chain proteins are at least 90% identical to SEQ ID NO: 3, and the recombinant immunoglobulin heavy chain protein comprises at least one of a substitution at the amino acid position corresponding to amino acid leucine (L) 234 of SEQ ID NO: 3, a substitution at the amino acid position corresponding to amino acid leucine (L) 235 of SEQ ID NO: 3, and a substitution at the amino acid position corresponding to amino asparagine (N) 297 of SEQ ID NO: 3. In some embodiments the sequences of the recombinant immunoglobulin heavy chain proteins are at least 90% identical to SEQ ID NO: 3, and the recombinant immunoglobulin heavy chain protein comprises at least one of a leucine (L) to alanine (A) substitution at the amino acid position corresponding to amino acid 234 of SEQ ID NO: 3, a leucine (L) to alanine (A) substitution at the amino acid position corresponding to amino acid 235 of SEQ ID NO: 3, and an asparagine (N) to alanine (A) substitution at the amino acid position corresponding to amino acid 297 of SEQ ID NO: 3. The sequence of the recombinant immunoglobulin heavy chain protein may be at least 95% identical to SEQ ID NO: 3. The sequence of the recombinant immunoglobulin heavy chain protein may comprise the amino acid sequence of SEQ ID NO: 1.

The sequence of the recombinant immunoglobulin heavy chain protein may comprise a leucine (L) to alanine (A) substitution at the amino acid position corresponding to amino acid 234 of SEQ ID NO: 3, and a leucine (L) to alanine (A) substitution at the amino acid position corresponding to amino acid 235 of SEQ ID NO: 3. The sequence of the recombinant immunoglobulin heavy chain protein may comprise or consist of SEQ ID NO: 4.

The recombinant immunoglobulin heavy chain protein may comprise an asparagine (N) to alanine (A) substitution at the amino acid position corresponding to amino acid 297 of SEQ ID NO: 3. The sequence of the recombinant immunoglobulin heavy chain protein may comprise or consist of SEQ ID NO: 5.

In another aspect this invention provides recombinant antibodies that comprise a recombinant immunoglobulin heavy chain protein and a recombinant immunoglobulin light chain protein.

In some embodiments of the recombinant antibodies the sequence of the recombinant immunoglobulin heavy chain protein is at least 90% identical to SEQ ID NO: 3, and the sequence of the recombinant immunoglobulin heavy chain protein comprises at least one substitution selected in the group consisting in leucine (L) to alanine (A) substitution at the amino acid position corresponding to amino acid 234 of SEQ ID NO: 3, a leucine (L) to alanine (A) substitution at the amino acid position corresponding to amino acid 235 of SEQ ID NO: 3, and an asparagine (N) to alanine (A) substitution at the amino acid position corresponding to amino acid 297 of SEQ ID NO: 3. In some embodiments of the recombinant antibodies the sequence of the recombinant immunoglobulin heavy chain protein may be at least 95% identical to SEQ ID NO: 3. In some embodiments of the recombinant antibodies the sequence of the recombinant immunoglobulin heavy chain protein may comprise the amino acid sequence of SEQ ID NO: 1.

In some embodiments of the recombinant antibodies the sequence of the recombinant immunoglobulin heavy chain protein may comprise a leucine (L) to alanine (A) substitution at the amino acid position corresponding to amino acid 234 of SEQ ID NO: 3, and a leucine (L) to alanine (A) substitution at the amino acid position corresponding to amino acid 235 of SEQ ID NO: 3. In some embodiments of the recombinant antibodies the sequence of the recombinant immunoglobulin heavy chain protein may comprise or consist of SEQ ID NO: 4.

In some embodiments of the recombinant antibodies the recombinant immunoglobulin heavy chain protein may comprise an asparagine (N) to alanine (A) substitution at the amino acid position corresponding to amino acid 297 of SEQ ID NO: 3. In some embodiments of the recombinant antibodies the sequence of the recombinant immunoglobulin heavy chain protein may comprise or consist of SEQ ID NO: 5.

In some embodiments of the recombinant antibodies the sequence of the recombinant immunoglobulin light chain proteins are at least 90% identical to SEQ ID NO: 6. In some embodiments of the recombinant antibodies the sequence of the recombinant immunoglobulin light chain protein may be at least 95% identical to SEQ ID NO: 6. In some embodiments of the recombinant antibodies the sequence of the recombinant immunoglobulin light chain protein may comprise the amino acid sequence of SEQ ID NO: 2. In some embodiments of the recombinant antibodies the sequence of the recombinant immunoglobulin light chain protein may comprise or consist of SEQ ID NO: 6.

In some embodiments of the recombinant antibodies the recombinant antibody blocks human IgE and IgE-mediated reactions at least 90% as effectively as omalizumab.

In some embodiments of the recombinant antibodies immune complexes of the recombinant antibody and human IgE bind to activating human FcγRs with an affinity that is reduced by at least 90% relative to omalizumab.

In some embodiments of the recombinant antibodies the recombinant antibody binds to human C1q with an affinity that is reduced by at least 90% relative to omalizumab.

In some embodiments of the recombinant antibodies induction of anaphylaxis following injection of immune complexes of the recombinant antibody bound to human IgE into humanized hFcγRKI mice (Beutier H, et al. Science Immunology, in press. Apr. 13, 2018) is reduced by at least 90% relative to omalizumab.

In some embodiments the recombinant antibody comprises a recombinant immunoglobulin heavy chain protein whose sequence is at least 90% identical to SEQ ID NO: 3 and a recombinant immunoglobulin light chain protein whose sequence is at least 90% identical to SEQ ID NO: 6; and the recombinant antibody blocks human IgE and IgE-mediated reactions at least 90% as effectively as omalizumab. In some embodiments immune complexes of the recombinant antibody and human IgE bind to activating human FcγRs with an affinity that is reduced by at least 90% relative to omalizumab. In some embodiments the recombinant antibody binds to human C1q with an affinity that is reduced by at least 90% relative to omalizumab. In some embodiments induction of anaphylaxis following injection of immune complexes of the recombinant antibody bound to human IgE into humanized hFcγRKI mice is reduced by at least 90% relative to omalizumab.

In another aspect this invention provides hybrid molecules comprising a first portion that is a Fab fragment of a recombinant antibody of the invention and a second portion that is a molecule that does not bind human FcγRs and/or does not bind human C1q. In some embodiments of the hybrid molecule the second portion is a Fab fragment of an anti-albumin antibody. In some embodiments of the hybrid molecule the second portion is polyethylene glycol. In some embodiments of the hybrid molecule the first portion is a Fab fragment omalizumab and the second portion that is a molecule that does not bind human FcγRs and/or does not bind human C1q. In some embodiments of the hybrid molecule the second portion is a Fab fragment of an anti-albumin antibody. In some embodiments of the hybrid molecule the second portion is polyethylene glycol.

In another aspect this invention provides nucleic acid molecules comprising a nucleotide sequence encoding a recombinant immunoglobulin heavy chain protein of the invention.

In another aspect this invention provides nucleic acid molecules and sets of nucleic acid molecules that comprise nucleotide sequences encoding a recombinant antibody of the invention or a hybrid molecule of the invention.

In another aspect this invention provides vectors and sets of vectors comprising a nucleic acid molecule or set of nucleic acid molecules of the invention.

In another aspect this invention provides recombinant host cells comprising a vector or set of vectors of the invention.

In another aspect this invention provides uses of a nucleic acid of the invention, a vector or set of vectors of the invention, or a recombinant host cell of the invention for producing a recombinant antibody of the invention or a hybrid molecule of the invention.

In another aspect this invention provides processes for producing a recombinant antibody of the invention or a hybrid molecule of the invention. The processes may comprise providing a recombinant host cell of the invention and culturing the host cell under conditions allowing expression of the recombinant antibody or hybrid molecule of the invention. In some embodiments the process further comprises recovering the recombinant antibody or hybrid molecule from the culture.

In another aspect this invention provides compositions comprising a recombinant antibody of the invention or a hybrid molecule of the invention and a carrier suitable for administration to a subject.

In another aspect this invention provides methods of reducing an IgE-mediated allergic response in a subject, comprising administering a recombinant antibody of the invention or a hybrid molecule of the invention to the subject.

In another aspect this invention provides methods of preventing or treating an IgE-mediated disease in a subject, comprising administering a recombinant antibody of the invention or a hybrid molecule of the invention to the subject. In some embodiments the IgE-mediated disease is selected from asthma and chronic idiopathic urticaria.

In another aspect this invention provides the use of a recombinant antibody of the invention or a hybrid molecule of the invention for preventing or treating an IgE-mediated disease in a subject.

In a particular embodiment, the invention is described by its claims.

### DETAILED DESCRIPTION OF THE INVENTION

The examples provided herein present data of the inventors' relating to making new human anti-IgE antibodies. Particularly, the inventors fused the omalizumab variable heavy chain domain (SEQ ID NO: 1) to a human Igγ1 constant domain to create a “starting” heavy chain molecule (SEQ ID NO: 3) and then demonstrated that is possible to make targetted amino acid changes to this heavy chain molecule in order to create new heavy chain molecules (SEQ ID NOS: 4 and 5) having useful properties when incorporated into a recombinant antibody.

The presented data demonstrates that a recombinant antibody having heavy chains with the amino acid sequence of SEQ ID NO: 3 and light chains with the amino acid sequence of SEQ ID NO: 6 is able to bind human IgE and to block IgE and IgE-mediated reactions similarly to omalizumab. The data also shows that immune complexes of the recombinant antibody and human IgE bind to activating human FcγRs in a manner similar to omalizumab that the recombinant antibody binds to human C1q in a manner similar to omalizumab.

The data also demonstrates that changes to the amino acid sequence of the heavy chain molecule result in recombinant antibodies that have reduced side effects. A first modified heavy chain molecule has the amino acid sequence of SEQ ID NO: 4. SEQ ID NO: 4 is the same as SEQ ID NO: 3 except that leucine (L) 234 is changed to alanine (A) and leucine (L) 235 is changed to alanine (A) (L234A, L235A).

A second modified heavy chain molecule has the amino acid sequence of SEQ ID NO: 5. SEQ ID NO: 5 is the same as SEQ ID NO: 3 except that asparagine (N) 297 is changed to alanine (A) (N297A).

The presented data demonstrates that a recombinant antibody having heavy chains with the amino acid sequence of SEQ ID NO: 4 and light chains with the amino acid sequence of SEQ ID NO: 6 is able to bind human IgE and to block IgE and IgE-mediated reactions similarly to omalizumab and similar to the inventors' starting antibody. The data also shows that immune complexes of the recombinant antibody and human IgE have significantly reduced binding to activating human FcγRs, that the recombinant antibody has significantly reduced binding to human C1q, and that injecting the recombinant antibody into humanized hFcγRKI mice has a significantly reduced ability to induce anaphylaxis. Taken together these data demonstrate that the recombinant antibody having heavy chains with the amino acid sequence of SEQ ID NO: 4 and light chains with the amino acid sequence of SEQ ID NO: 6 is efficacious with significantly reduced side effects.

The presented data further demonstrates that a recombinant antibody having heavy chains with the amino acid sequence of SEQ ID NO: 5 and light chains with the amino acid sequence of SEQ ID NO: 6 is able to bind human IgE and to block IgE and IgE-mediated reactions similarly to omalizumab and similar to the inventors' starting antibody. The data also shows that immune complexes of the recombinant antibody and human IgE have significantly reduced binding to activating human FcγRs, that the recombinant antibody has significantly reduced binding to human C1q, and that injecting the recombinant antibody into humanized hFcγRKI mice has a significantly reduced ability to induce anaphylaxis. Taken together these data demonstrate that the recombinant antibody having heavy chains with the amino acid sequence of SEQ ID NO: 5 and light chains with the amino acid sequence of SEQ ID NO: 6 is efficacious with significantly reduced side effects.

As used herein, the “percent identity” or the percent to which two sequences are “identical” is calculated following allignment of the two sequences. The alignment may be accomplished by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and the scoring matrix PAM 250 [a standard scoring matrix; see Dayhoff et al., in Atlas of Protein Sequence and Structure, vol. 5, supp. 3 (1978)] is used in conjunction with the computer program. The percent identity is then calculated as: the total number of identical matches multiplied by 100 and then divided by the sum of the length of the longer sequence within the matched span and the number of gaps introduced into the shorter sequences in order to align the two sequences.

A. Mutations and Recombinant Immunoglobulin Heavy Chains

In a particular embodiement, the recombinant immunoglobulin heavy chains protein can comprises at least one mutation in the Fc portion. These mutations can be selected in the groups consisting in:

Starting Mutations: L234A, L235A, N297A

Mutations inducing reduced binding to all FcγR: E233P, L234V, G236 deleted, P238A, D265A, A327Q, P329A

Mutations inducing reduced binding to FcγRII and FcγRIIIA D270A, Q295A, A327S

Mutations inducing reduced binding to FcγRII and no effect on FcγRIIIA R292A, K414A

Mutations inducing no effect on FcγRII and reduced binding to FcγRI11A S239A, E269A, E293A, Y296F V303A, A327G, K338A D376A

Mutations affecting only FcRn: I253A, S254A, K288A, V305A, Q311A, D312A, K317A, K360A, Q362A, E380A, E382A, S415A, S424A, H433A, N434A, H435A, Y436A

Mutations inducing increasing C1q binding: K326W, E333S, S267E, H268F, S324T

Mutations inducing hexamerization: E345R, E430G, S440Y

Mutations inducing aglycosylation: N297Q, N297G

Mutations inducing reduced FcγR and C1q binding: L235E, F234A, H268Q, V309L, A330S, P331S, V234A, G237A, P238S, S298N, K322A, L234F, L328F.

The nomenclature of these mutations is obtained thanks to the EU numbering (see for example http://www.imgt.org/IMGTScientificChart/Numbering/Hu_IGHGnber.html#notes or Edelman G M et al., 1969).

Thus, this invention also provides recombinant immunoglobulin heavy chain proteins which comprises at least one mutation in the Fc portion. Particularly, the mutations are described below.

According to the invention, the recombinant immunoglobulin heavy chain protein can also be derived and obtained from the immunoglobulin heavy chain protein of the omalizumab, the Ligelizumab, the Quilizumab, the XmAb7195 (see for example the patent application US2017247470 for the sequences), the MEDI4212 (see for example Balbino B. et al, Approaches to target IgE antibodies in allergic diseases, Pharmacology & Therapeutics 2018).

In a particular embodiement, the recombinant immunoglobulin heavy chain protein can be derived and obtained from the variants of the Omalizumab as described in the patent application WO2017211928.

According to the invention, the sequences of the Ligelizumab are:

Heavy chain sequence of the Ligelizumab
(SEQ ID NO: 8):
QVQLVQSGAE VMKPGSSVKV SCKASGYTFS WYWLEWVRQA
PGHGLEWMGE IDPGTFTTNY NEKFKARVTF TADTSTSTAY
MELSSLRSED TAVYYCARFS HFSGSNYDYF DYWGQGTLVT
VSSASTKGPS VFPLAPSSKS TSGGTAALGC LVKDYFPEPV
TVSWNSGALT SGVHTFPAVL QSSGLYSLSS VVTVPSSSLG
TQTYICNVNH KPSNTKVDKK VEPKSCDKTH TCPPCPAPEL
LGGPSVFLFP PKPKDTLMIS RTPEVTCVVV DVSHEDPEVK
FNWYVDGVEV HNAKTKPREE QYNSTYRVVS VLTVLHQDWL
NGKEYKCKVS NKALPAPIEK TISKAKGQPR EPQVYTLPPS
RDELTKNQVS LTCLVKGFYP SDIAVEWESN GQPENNYKTT
PPVLDSDGSF FLYSKLTVDK SRWQQGNVFS CSVMHEALHN
HYTQKSLSLS PGK
Light chain sequence of the Ligelizumab
(SEQ ID NO: 9):
EIVMTQSPAT LSVSPGERAT LSCRASQSIG THIHWYQQKP
GQAPRLLIYY ASESISGIPA RFSGSGSGTE FTLTISSLQS
EDFAVYYCQQ SWSWPTTFGG GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

According to the invention, the sequences of the Quilizumab are:

Heavy chain sequence of the Quilizumab
(SEQ ID NO: 10):
EVQLVESGGG LVQPGGSLRL SCAASGFTFS DYGIAWVRQA
PGKGLEWVAF ISDLAYTIYY ADTVTGRFTI SRDNSKNTLY
LQMNSLRAED TAVYYCARDN WDAMDYWGQG TLVTVSSAST
KGPSVFPLAP SSKSTSGGTA ALGCLVKDYF PEPVTVSWNS
GALTSGVHTF PAVLQSSGLY SLSSVVTVPS SSLGTQTYIC
NVNHKPSNTK VDKKVEPKSC DKTHTCPPCP APELLGGPSV
FLFPPKPKDT LMISRTPEVT CVVVDVSHED PEVKFNWYVD
GVEVHNAKTK PREEQYNSTY RVVSVLTVLH QDWLNGKEYK
CKVSNKALPA PIEKTISKAK GQPREPQVYT LPPSREEMTK
NQVSLTCLVK GFYPSDIAVE WESNGQPENN YKTTPPVLDS
DGSFFLYSKL TVDKSRWQQG NVFSCSVMHE ALHNHYTQKS
LSLSPGK
Light chain sequence of the Quilizumab
(SEQ ID NO: 11):
DIQMTQSPSS LSASVGDRVT ITCRSSQSLV HNNANTYLHW
YQQKPGKAPK LLIYKVSNRF SGVPSRFSGS GSGTDFTLTI
SSLQPEDFAT YYCSQNTLVP WTFGQGTKVE IKRTVAAPSV
FIFPPSDEQL KSGTASVVCL LNNFYPREAK VQWKVDNALQ
SGNSQESVTE QDSKDSTYSL SSTLTLSKAD YEKHKVYACE
VTHQGLSSPV TKSFNRGEC

Concerning the ligelizumab, the VH sequence, contain six (6) supplemental amino acids compared for example to the omalizumab, so all the mutations as described in the invention are shifted of six (6) aminos acids. Thus, for example the mutations L234A, L235A and N297A for the Omalizumab are L240A, L241A, N303A for the ligelizumab. Thus, for the ligelizumab, all mutations of the invention in the immunoglobulin heavy chain protein are shifted of 6 amino acids.

In some embodiments the recombinant immunoglobulin heavy chain proteins are variants of SEQ ID NO: 3, 8 or 10 that are at least 90%, at least 91%, at least 92%, at least 93%, at least 94%, at least 95%, at least 96%, at least 97%, at least 98%, or at least 99% identical to SEQ ID NO: 3, 8 or 10. In some embodiments the recombinant immunoglobulin heavy chain proteins are variants of SEQ ID NO: 3, 8 or 10 that are 90%, 91%, 92%, 93%, 94%, 95%, 96%, 97%, 98%, or 99% identical to SEQ ID NO: 3, 8 or 10.

In some embodiments the recombinant immunoglobulin heavy chain protein comprises the amino acid sequence of SEQ ID NO: 1.

SEQ ID NO: 1 is the omalizumab variable heavy chain domain. SEQ ID NO: 1 is present at the amino terminus of SEQ ID NO: 3. In some embodiments the recombinant immunoglobulin heavy chain proteins comprise SEQ ID NO: 1. Thus, in such embodiments the amino acid difference(s) between SEQ ID NO: 3 and the recombinant immunoglobulin heavy chain proteins are at amino acid positions outside of the SEQ ID NO: 1 domain of SEQ ID NO: 3.

These recombinant immunoglobulin heavy chain proteins can comprise at leat one mutation as described above.

In some embodiments the sequences of the recombinant immunoglobulin heavy chain proteins are at least 90% identical to SEQ ID NO: 3 or 10, and the recombinant immunoglobulin heavy chain protein comprises at least one substitution selected in the group consisting in a substitution at the amino acid position corresponding to amino acid leucine (L) 234 of SEQ ID NO: 3 or 10, a substitution at the amino acid position corresponding to amino acid leucine (L) 235 of SEQ ID NO: 3 or 10, and a substitution at the amino acid position corresponding to amino asparagine (N) 297 of SEQ ID NO: 3 or 10. In some embodiments the sequences of the recombinant immunoglobulin heavy chain protein comprises at least one substitution selected in the group consisting in a leucine (L) to alanine (A) substitution at the amino acid position corresponding to amino acid 234 of SEQ ID NO: 3 or 10, a leucine (L) to alanine (A) substitution at the amino acid position corresponding to amino acid 235 of SEQ ID NO: 3 or 10, and an asparagine (N) to alanine (A) substitution at the amino acid position corresponding to amino acid 297 of SEQ ID NO: 3 or 10. The sequences of the recombinant immunoglobulin heavy chain protein may be at least 95% identical to SEQ ID NO: 3 or 10.

In some embodiments any of the sequences of the recombinant immunoglobulin heavy chain proteins of this invention may comprise a mutation or set of mutations at an amino acid of SEQ ID NO: 3 or 10 corresponding to a mutation or set of mutations listed in Table 5 at page 44 of Bruhns, et al., “Mouse and human FcR effector functions,” Immunological Reviews 2015 Vol. 268: 25-51, which is hereby incorporated by reference.

In some embodiments the recombinant immunoglobulin heavy chain protein may comprise a leucine (L) to alanine (A) substitution at the amino acid position corresponding to amino acid 234 of SEQ ID NO: 3 or 10, and a leucine (L) to alanine (A) substitution at the amino acid position corresponding to amino acid 235 of SEQ ID NO: 3 or 10.

In some embodiments the sequence of the recombinant immunoglobulin heavy chain protein comprises SEQ ID NO: 4. In some embodiments the sequence of the recombinant immunoglobulin heavy chain protein consists of SEQ ID NO: 4.

In some embodiments the recombinant immunoglobulin heavy chain protein may comprise an asparagine (N) to alanine (A) substitution at the amino acid position corresponding to amino acid 297 of SEQ ID NO: 3 or 10.

In some embodiments the sequence of the recombinant immunoglobulin heavy chain protein comprises SEQ ID NO: 5. In some embodiments the sequence of the recombinant immunoglobulin heavy chain protein consists of SEQ ID NO: 5.

In some embodiments the sequences of the recombinant immunoglobulin heavy chain proteins are at least 90% identical to SEQ ID NO: 8, and the recombinant immunoglobulin heavy chain protein comprises at least one of a substitution selected in the group consisting ina substitution at the amino acid position corresponding to amino acid leucine (L) 240 of SEQ ID NO: 8, a substitution at the amino acid position corresponding to amino acid leucine (L) 241 of SEQ ID NO: 8, and a substitution at the amino acid position corresponding to amino asparagine (N) 303 of SEQ ID NO: 8. In some embodiments the sequences of the recombinant immunoglobulin heavy chain protein comprises at least one substitution selected in the group consisting in a leucine (L) to alanine (A) substitution at the amino acid position corresponding to amino acid 240 of SEQ ID NO: 8, a leucine (L) to alanine (A) substitution at the amino acid position corresponding to amino acid 241 of SEQ ID NO: 8, and an asparagine (N) to alanine (A) substitution at the amino acid position corresponding to amino acid 303 of SEQ ID NO: 8 or 10. The sequences of the recombinant immunoglobulin heavy chain protein may be at least 95% identical to SEQ ID NO: 8.

In some embodiments the recombinant immunoglobulin heavy chain protein may comprise a leucine (L) to alanine (A) substitution at the amino acid position corresponding to amino acid 240 of SEQ ID NO: 8, and a leucine (L) to alanine (A) substitution at the amino acid position corresponding to amino acid 241 of SEQ ID NO: 8.

In some embodiments the recombinant immunoglobulin heavy chain protein may comprise an asparagine (N) to alanine (A) substitution at the amino acid position corresponding to amino acid 303 of SEQ ID NO: 8.

B. Recombinant Antibodies

As used herein, an “antibody” is a molecule that consists of four polypeptide chains, two identical immunoglobulin heavy chains and two identical light chains.

This invention also provides recombinant antibodies.

In a particular embodiement, the recombinant antibody is an anti-IgE recombinant antibody.

In another particular embodiement, the recombinant antibody is an anti-IgE recombinant antibody with a recombinant immunoglobulin heavy chain protein according to the invention which comprises at least one mutation in the Fc portion.

In a particular embodiement, the recombinant antibody is an IgG1, IgG2, IgG3 or IgG4. More particularly, the anti-IgE antibody is an IgG1 antibody.

In a particular embodiement, the invention relates to an anti-IgE recombinant antibody with a recombinant immunoglobulin heavy chain protein according to the invention which binds an epitope comprising residues T373, W374, 5375, R376, A377, 5378, G379, P381, Q417, C418, R419, T421, P426, R427, A428 of a CE3 domain and residues D278 and T281 of a Cs2 domain of human IgE of SEQ ID NO: 7. In further embodiments the epitope may further comprise one or more of residues K380 and/or M430 of the Cs3 domain of human IgE and/or one or more of residues D276, V277, L279, 5280, A282 and/or T298 of the Cs2 domain of human IgE.

SEQ ID NO: 7 (Wild-type human IgE-Fc (CE2-CE4 domains with numbering V224-K547 according to Dorrington & Bennich (1978)):

VASRDFTPPTVKILQSSCDGGGHFPPTIQLLCLVSGYTPGINITWLEDGQ
VMDVDLSTASTTQEGELASTQSELTLSQKHWLSDRTYTCQVTYQGHTFED
STKKCADSNPRGVSAYLSRPSPFDLFIRKSPTITCLVVDLAPSKGTVNLT
WSRASGKPVNHSTRKEEKQRNGTLTVTSTLPVGTRDWIEGETYQCRVTHP
HLPRALMRSTTKTSGPRAAPEVYAFATPEWPGSRDKRTLACLIQNFMPED
ISVQWLHNEVQLPDARHSTTQPRKTKGSGFFVFSRLEVTRAEWEQKDEFI
CRAVHEAASPSQTVQRAVSVNPGK

The recombinant antibodies may comprise any of the recombinant immunuglobulin heavy chain proteins of the invention. In some embodiments the recombinant antibody comprises a recombinant immunoglobulin heavy chain protein whose sequence is a variant of SEQ ID NO: 3, 8 or 10 that is at least 90%, at least 91%, at least 92%, at least 93%, at least 94%, at least 95%, at least 96%, at least 97%, at least 98%, or at least 99% identical to SEQ ID NO: 3, 8 or 10. In some embodiments the recombinant antibody comprises a recombinant immunoglobulin heavy chain protein whose sequence is a variant of SEQ ID NO: 3, 8 or 10 that is 90%, 91%, 92%, 93%, 94%, 95%, 96%, 97%, 98%, or 99% identical to SEQ ID NO: 3, 8 or 10.

In some embodiments the recombinant antibody comprises a recombinant immunoglobulin heavy chain protein whose sequences comprises the amino acid sequence of SEQ ID NO: 1.

SEQ ID NO: 1 represents the omalizumab variable heavy chain domain. SEQ ID NO: 1 is present at the amino terminus of SEQ ID NO: 3. In some embodiments the recombinant antibody comprises a recombinant immunoglobulin heavy chain protein that comprises SEQ ID NO: 1. Thus, in such embodiments the amino acid difference(s) between SEQ ID NO: 3 and the recombinant immunoglobulin heavy chain protein of the recombinant antibody are at amino acid positions outside of the SEQ ID NO: 1 domain of SEQ ID NO: 3.

In some embodiments the recombinant antibody comprises a recombinant immunoglobulin heavy chain protein that comprises at least one substitution selected in the group consisting in a leucine (L) to alanine (A) substitution at the amino acid position corresponding to amino acid 234 of SEQ ID NO: 3 or 10, a leucine (L) to alanine (A) substitution at the amino acid position corresponding to amino acid 235 of SEQ ID NO: 3 or 10, and an asparagine (N) to alanine (A) substitution at the amino acid position corresponding to amino acid 297 of SEQ ID NO: 3 or 10.

In some embodiments the recombinant antibody comprises a recombinant immunoglobulin heavy chain protein that comprises a recombinant immunoglobulin heavy chain protein comprising a leucine (L) to alanine (A) substitution at the amino acid position corresponding to amino acid 234 of SEQ ID NO: 3 or 10, and a leucine (L) to alanine (A) substitution at the amino acid position corresponding to amino acid 235 of SEQ ID NO: 3 or 10. In some embodiments the recombinant antibody comprises a recombinant immunoglobulin heavy chain protein that comprises SEQ ID NO: 4. In some embodiments the recombinant antibody comprises a recombinant immunoglobulin heavy chain protein that consists of SEQ ID NO: 4.

In some embodiments the recombinant antibody comprises a recombinant immunoglobulin heavy chain protein that comprises a recombinant immunoglobulin heavy chain protein comprising an asparagine (N) to alanine (A) substitution at the amino acid position corresponding to amino acid 297 of SEQ ID NO: 3 or 10. In some embodiments the recombinant antibody comprises a recombinant immunoglobulin heavy chain protein that comprises SEQ ID NO: 5. In some embodiments the recombinant antibody comprises a recombinant immunoglobulin heavy chain protein that consists of SEQ ID NO: 5.

In some embodiments the recombinant antibody comprises a recombinant immunoglobulin light chain protein that is at least 90%, at least 91%, at least 92%, at least 93%, at least 94%, at least 95%, at least 96%, at least 97%, at least 98%, or at least 99% identical to SEQ ID NO: 6. In some embodiments the recombinant antibody comprises a recombinant immunoglobulin light chain protein that is 90%, 91%, 92%, 93%, 94%, 95%, 96%, 97%, 98%, or 99% identical to SEQ ID NO: 6. In some embodiments the recombinant antibody comprises a recombinant immunoglobulin light chain protein that comprises the amino acid sequence of SEQ ID NO: 2.

SEQ ID NO: 2 represents the omalizumab variable light chain domain. SEQ ID NO: 2 is present at the amino terminus of SEQ ID NO: 6. In some embodiments the recombinant antibody comprises a recombinant immunoglobulin light chain protein whose sequence comprises SEQ ID NO: 2. Thus, in such embodiments any amino acid difference(s) between SEQ ID NO: 6 and the recombinant immunoglobulin light chain protein of the recombinant antibody are at amino acid positions outside of the SEQ ID NO: 2 domain of SEQ ID NO: 6.

In some embodiments of the recombinant antibodies the recombinant antibody blocks human IgE and IgE-mediated reactions at least 50%, 60%, 70%, 80%, 90%, or 95% as effectively as omalizumab. A suitable assay for measuring the blocking of human IgE and IgE-mediated reactions in comparison to omalizumab is that used in Example 7. In some embodiments of the recombinant antibodies the recombinant antibody blocks human IgE and IgE-mediated reactions at least as effectively as omalizumab, Ligelizumab or Quilizumab.

In some embodiments of the recombinant antibodies the recombinant antibody has an affinity constant (KA) for human IgE that is at least 20%, 30%, 40%, 50%, 60%, 70%, 80%, 90%, or 95% of that of omalizumab, Ligelizumab or Quilizumab when measured in the same assay. In some embodiments of the recombinant antibodies the recombinant antibody has an affinity constant (KA) for human IgE of at least 1×109 M-1, 2×109 M-1, 3×109 M-1, 4×109 M-1, 5×109 M-1, 6×109 M-1, 7×109 M-1, 8×109 M-1, or 1×109 M-1. In some embodiments of the recombinant antibodies the recombinant antibody has an affinity constant (KA) for human IgE of 1×109 M-1, 2×109 M-1, 3×109 M-1, 4×109 M-1, 5×109 M-1, 6×109 M-1, 7×109 M-1, 8×109 M-1, or 1×109 M-1.

In some embodiments of the recombinant antibodies the recombinant antibody blocks human IgE and IgE-mediated reactions 50%, 60%, 70%, 80%, 90%, or 95% as effectively as omalizumab, Ligelizumab or Quilizumab. In some embodiments of the recombinant antibodies the recombinant antibody blocks human IgE and IgE-mediated reactions as effectively as omalizumab, Ligelizumab or Quilizumab.

The recombinant antibodies of the invention bind to IgE to form immune complexes. It is these immune complexes that then bind to activating human FcγRs to cause undesirable side effects. In some embodiments of the recombinant antibodies immune complexes of the recombinant antibody and human IgE bind to activating human FcγRs with an affinity that is reduced by at least 20%, 30%, 40%, 50%, 60%, 70%, 80%, 90%, or 95% relative to omalizumab, Ligelizumab or Quilizumab. In some embodiments the affinity is reduced to an undetectable level. A suitable assay for measuring the activating of human FcγRs in comparison to omalizumab is that used in Example 5. In some embodiments of the recombinant antibodies immune complexes of the recombinant antibody and human IgE bind to activating human FcγRs with an affinity that is reduced by 20%, 30%, 40%, 50%, 60%, 70%, 80%, 90%, or 95% relative to omalizumab, Ligelizumab or Quilizumab.

In some embodiments of the recombinant antibodies the recombinant antibody binds to human C1q with an affinity that is reduced by at least 20%, 30%, 40%, 50%, 60%, 70%, 80%, 90%, or 95% relative to omalizumab. In some embodiments the affinity is reduced to an undetectable level. A suitable assay for measuring binding to human C1q in comparison to omalizumab is that used in Example 5. In some embodiments of the recombinant antibodies the recombinant antibody binds to human C1q with an affinity that is reduced by 20%, 30%, 40%, 50%, 60%, 70%, 80%, 90%, or 95% relative to omalizumab.

In some embodiments of the recombinant antibodies induction of anaphylaxis following injection of immune complexes of the recombinant antibody and human IgE into humanized hFcγRKI mice is reduced by at least 20%, 30%, 40%, 50%, 60%, 70%, 80%, 90%, or 95% relative to omalizumab, Ligelizumab or Quilizumab. A suitable assay for measuring induction of anaphylaxis following injection in comparison to omalizumab is that used in Example 8. In some embodiments induction of anaphylaxis is reduced to an undetectable level. In some embodiments of the recombinant antibodies induction of anaphylaxis following injection of immune complexes of the recombinant antibody and human IgE into humanized hFcγRKI mice is reduced by 20%, 30%, 40%, 50%, 60%, 70%, 80%, 90%, or 95% relative to omalizumab, Ligelizumab or Quilizumab.

The data provided in the examples demonstrate for the first time that it is possible to create recombinant antibodies that are variants of omalizumab and that (1) retain the ability to bind to human IgE in a manner comparable to omalizumab, but (2) have reduced side effects relative to omalizumab. Accordingly, the invention also enables and includes recombinant antibodies comprising an immunoglobulin heavy chain protein whose sequence differs from SEQ ID NO: 3 or 10 at one or more amino acid positions within the Fc portion of heavy chain that are other than L234, L235 and N297.

Accordingly, in some embodiments the recombinant antibody (1) comprises a recombinant immunoglobulin heavy chain protein whose sequence is at least 90%, 91%, 92%, 93%, 94%, 95%, 96%, 97%, 98%, or 99% identical to SEQ ID NO: 3, 8 or 10 and a recombinant immunoglobulin light chain protein that is at least 90%, 91%, 92%, 93%, 94%, 95%, 96%, 97%, 98%, or 99% identical to SEQ ID NO: 6, 9 or 11; (2) blocks human IgE and IgE-mediated reactions at least 90% as effectively as omalizumab; and (3) has at least one property selected from immune complexes of the recombinant antibody and human IgE bind to activating human FcγRs with an affinity that is reduced by at least 90% relative to omalizumab, Ligelizumab or Quilizumab; binds to human C1q with an affinity that is reduced by at least 90% relative to omalizumab, Ligelizumab or Quilizumab; induction of anaphylaxis following injection of immune complexes of the recombinant antibody bound to human IgE into humanized hFcγRK1 mice is reduced by at least 90% relative to omalizumab, Ligelizumab or Quilizumab.

In an embodiment the recombinant antibody of the invention comprises (1) a recombinant immunoglobulin heavy chain protein whose sequence is at least 97% identical to SEQ ID NO: 3 or 10 and comprises at least one substitution selected in the group consisting in a leucine (L) to alanine (A) substitution at the amino acid position corresponding to amino acid 234 of SEQ ID NO: 3 or 10, a leucine (L) to alanine (A) substitution at the amino acid position corresponding to amino acid 235 of SEQ ID NO: 3 or 10, and an asparagine (N) to alanine (A) substitution at the amino acid position corresponding to amino acid 297 of SEQ ID NO: 3 or 10; and (2) a recombinant immunoglobulin light chain protein whose sequence is at least 97% identical to SEQ ID NO: 6 or 11.

In an embodiment the recombinant antibody of the invention comprises (1) a recombinant immunoglobulin heavy chain protein whose sequence is at least 97% identical to SEQ ID NO: 3 or 10 and comprises a leucine (L) to alanine (A) substitution at the amino acid position corresponding to amino acid 234 of SEQ ID NO: 3 or 10 and a leucine (L) to alanine (A) substitution at the amino acid position corresponding to amino acid 235 of SEQ ID NO: 3 or 10; and (2) a recombinant immunoglobulin light chain protein whose sequence is at least 97% identical to SEQ ID NO: 6 or 11.

In an embodiment the recombinant antibody of the invention comprises (1) a recombinant immunoglobulin heavy chain protein whose sequence is at least 97% identical to SEQ ID NO: 3 or 10 and comprises an asparagine (N) to alanine (A) substitution at the amino acid position corresponding to amino acid 297 of SEQ ID NO: 3 or 10; and (2) a recombinant immunoglobulin light chain protein whose sequence is at least 97% identical to SEQ ID NO: 6 or 11.

In an embodiment the recombinant antibody of the invention comprises (1) a recombinant immunoglobulin heavy chain protein whose sequence comprises SEQ ID NO: 1, is at least 97% identical to SEQ ID NO: 3, and comprises at least one substitution selected in the group consisting in a leucine (L) to alanine (A) substitution at the amino acid position corresponding to amino acid 234 of SEQ ID NO: 3, a leucine (L) to alanine (A) substitution at the amino acid position corresponding to amino acid 235 of SEQ ID NO: 3, and an asparagine (N) to alanine (A) substitution at the amino acid position corresponding to amino acid 297 of SEQ ID NO: 3; and (2) a recombinant immunoglobulin light chain protein whose sequence comprises SEQ ID NO: 2 is at least 97% identical to SEQ ID NO: 6.

In an embodiment the recombinant antibody of the invention comprises (1) a recombinant immunoglobulin heavy chain protein whose sequence comprises SEQ ID NO: 1, is at least 97% identical to SEQ ID NO: 3 and comprises a leucine (L) to alanine (A) substitution at the amino acid position corresponding to amino acid 234 of SEQ ID NO: 3 and a leucine (L) to alanine (A) substitution at the amino acid position corresponding to amino acid 235 of SEQ ID NO: 3; and (2) a recombinant immunoglobulin light chain protein whose sequence comprises SEQ ID NO: 2 is at least 97% identical to SEQ ID NO: 6.

In an embodiment the recombinant antibody of the invention comprises (1) a recombinant immunoglobulin heavy chain protein whose sequence comprises SEQ ID NO: 1, is at least 97% identical to SEQ ID NO: 3 and comprises an asparagine (N) to alanine (A) substitution at the amino acid position corresponding to amino acid 297 of SEQ ID NO: 3; and (2) a recombinant immunoglobulin light chain protein whose sequence comprises SEQ ID NO: 2 is at least 97% identical to SEQ ID NO: 6.

In an embodiment the recombinant antibody of the invention comprises (1) a recombinant immunoglobulin heavy chain protein whose sequence comprises SEQ ID NO: 4; and (2) a recombinant immunoglobulin light chain protein whose sequence comprises SEQ ID NO: 6.

In an embodiment the recombinant antibody of the invention comprises (1) a recombinant immunoglobulin heavy chain protein whose sequence consists of SEQ ID NO: 4; and (2) a recombinant immunoglobulin light chain protein whose sequence consists of SEQ ID NO: 6.

In some embodiments the recombinant antibody comprises a recombinant immunoglobulin heavy chain protein that comprises at least one substitution selected in the group consisting in a leucine (L) to alanine (A) substitution at the amino acid position corresponding to amino acid 240 of SEQ ID NO: 8, a leucine (L) to alanine (A) substitution at the amino acid position corresponding to amino acid 241 of SEQ ID NO: 8, and an asparagine (N) to alanine (A) substitution at the amino acid position corresponding to amino acid 303 of SEQ ID NO: 8.

In an embodiment the recombinant antibody of the invention comprises (1) a recombinant immunoglobulin heavy chain protein whose sequence is at least 97% identical to SEQ ID NO: 8 and comprises at least one substitution selected in the group consisting in a leucine (L) to alanine (A) substitution at the amino acid position corresponding to amino acid 240 of SEQ ID NO: 8, a leucine (L) to alanine (A) substitution at the amino acid position corresponding to amino acid 241 of SEQ ID NO: 8, and an asparagine (N) to alanine (A) substitution at the amino acid position corresponding to amino acid 303 of SEQ ID NO: 8; and (2) a recombinant immunoglobulin light chain protein whose sequence is at least 97% identical to SEQ ID NO: 9.

In an embodiment the recombinant antibody of the invention comprises (1) a recombinant immunoglobulin heavy chain protein whose sequence is at least 97% identical to SEQ ID NO: 8 and comprises a leucine (L) to alanine (A) substitution at the amino acid position corresponding to amino acid 240 of SEQ ID NO: 8 and a leucine (L) to alanine (A) substitution at the amino acid position corresponding to amino acid 241 of SEQ ID NO: 8; and (2) a recombinant immunoglobulin light chain protein whose sequence is at least 97% identical to SEQ ID NO: 9.

In an embodiment the recombinant antibody of the invention comprises (1) a recombinant immunoglobulin heavy chain protein whose sequence is at least 97% identical to SEQ ID NO: 8 and comprises an asparagine (N) to alanine (A) substitution at the amino acid position corresponding to amino acid 303 of SEQ ID NO: 8; and (2) a recombinant immunoglobulin light chain protein whose sequence is at least 97% identical to SEQ ID NO: 9.

Cross-Competition

The invention also relates to a recombinant antibody which cross-competes for binding to IgE with a recombinant antibody comprising a recombinant immunoglobulin heavy chain protein which comprises at least one mutation in the Fc portion according to the invention.

In a particular embodiment, the invention also relates to a recombinant antibody which cross-competes for binding to IgE with a recombinant antibody comprising a recombinant immunoglobulin heavy chain protein comprising at least one substitution selected in the group consisting in a substitution at the amino acid position corresponding to amino acid leucine (L) 234 of SEQ ID NO: 3, a substitution at the amino acid position corresponding to amino acid leucine (L) 235 of SEQ ID NO: 3, and a substitution at the amino acid position corresponding to amino asparagine (N) 297 of SEQ ID NO: 3.

In another particular embodiment, the invention also relates to a recombinant antibody which cross-competes for binding to IgE with a recombinant antibody comprising a recombinant immunoglobulin heavy chain protein comprising at least one substitution selected in the group consisting in a substitution at the amino acid position corresponding to amino acid leucine (L) 240 of SEQ ID NO: 8, a substitution at the amino acid position corresponding to amino acid leucine (L) 241 of SEQ ID NO: 8, and a substitution at the amino acid position corresponding to amino asparagine (N) 303 of SEQ ID NO: 8.

In another particular embodiment, the invention also relates to a recombinant antibody which cross-competes for binding to IgE with a recombinant antibody comprising a recombinant immunoglobulin heavy chain protein comprising at least one substitution selected in the group consisting in a substitution at the amino acid position corresponding to amino acid leucine (L) 234 of SEQ ID NO: 10, a substitution at the amino acid position corresponding to amino acid leucine (L) 235 of SEQ ID NO: 10, and a substitution at the amino acid position corresponding to amino asparagine (N) 297 of SEQ ID NO: 10.

In a particular embodiment, the recombinant antibody which cross-competes for binding to IgE is anti-IgE recombinant antibody according to the invention.

As used herein, the term “cross-competes” refers to monoclonal antibodies which share the ability to bind to a specific region of an antigen. In the present disclosure the monoclonal antibody that “cross-competes” has the ability to interfere with the binding of another monoclonal antibody of the invention for the antigen in a standard competitive binding assay. Such a monoclonal antibody may, according to non-limiting theory, bind to the same or a related or nearby (e.g., a structurally similar or spatially proximal) epitope as the antibody with which it competes. Cross-competition is present if antibody A reduces binding of antibody B at least by 60%, specifically at least by 70% and more specifically at least by 80% and vice versa in comparison to the positive control which lacks one of said antibodies. As the skilled artisan appreciates competition may be assessed in different assay set-ups. One suitable assay involves the use of the Biacore technology (e.g., by using the BIAcore 3000 instrument (Biacore, Uppsala, Sweden)), which can measure the extent of interactions using surface plasmon resonance technology. Another assay for measuring cross-competition uses an ELISA-based approach. Furthermore, a high throughput process for “binning” antibodies based upon their cross-competition is described in International Patent Application No. WO2003/48731.

As used herein, the term “binding” in the context of the binding of an antibody to a predetermined antigen or epitope typically is a binding with an affinity corresponding to a K D of about 10⁻⁷ M or less, such as about 10⁻⁸ M or less, such as about 10⁻⁹ M or less, about 10⁻¹⁰ M or less, or about 10⁻¹¹ M or even less when determined by for instance surface plasmon resonance (SPR) technology in a BIAcore 3000 instrument using a soluble form of the antigen as the ligand and the antibody as the analyte. BIACORE® (GE Healthcare, Piscaataway, NJ) is one of a variety of surface plasmon resonance assay formats that are routinely used to epitope bin panels of monoclonal antibodies. Typically, an antibody binds to the predetermined antigen with an affinity corresponding to a K D that is at least ten-fold lower, such as at least 100-fold lower, for instance at least 1,000-fold lower, such as at least 10,000-fold lower, for instance at least 100,000-fold lower than its K D for binding to a non-specific antigen (e.g., BSA, casein), which is not identical or closely related to the predetermined antigen. When the K D of the antibody is very low (that is, the antibody has a high affinity), then the K D with which it binds the antigen is typically at least 10,000-fold lower than its K D for a non-specific antigen. An antibody is said to essentially not bind an antigen or epitope if such binding is either not detectable (using, for example, plasmon resonance (SPR) technology in a BIAcore 3000 instrument using a soluble form of the antigen as the ligand and the antibody as the analyte), or is 100 fold, 500 fold, 1000 fold or more than 1000 fold less than the binding detected by that antibody and an antigen or epitope having a different chemical structure or amino acid sequence.

Additional antibodies can be identified based on their ability to cross-compete (e.g., to competitively inhibit the binding of, in a statistically significant manner) with other antibodies of the invention in standard IgE binding assays. The ability of a test antibody to inhibit the binding of antibodies of the present invention to IgE demonstrates that the test antibody can compete with that antibody for binding to IgE; such an antibody may, according to non-limiting theory, bind to the same or a related (e.g., a structurally similar or spatially proximal) epitope on IgE as the antibody with which it competes. Thus, another aspect of the invention provides antibodies that bind to the same antigen as, and compete with, the antibodies disclosed herein. As used herein, an antibody “competes” for binding when the competing antibody inhibits IgE binding of an antibody or antigen binding fragment of the invention by more than 50,51,52,53,54,55,56,57,58,59,60,61,62,63,64,65,66,67,68,69,70,71,72,73,74,75,76,77,78,79, 80,81,82,83,84,85,86,87,88,89,90,91,92,93,94,95,96,97,98 or 99% in the presence of an equimolar concentration of competing antibody.

In other embodiments the antibodies or antigen binding fragments of the invention bind to one or more epitopes of IgE. In some embodiments, the epitopes to which the present antibodies or antigen binding fragments bind are linear epitopes. In other embodiments, the epitopes to which the present antibodies or antigen binding fragments bind are non-linear, conformational epitopes. In a particular embodiement the epitope comprises residues T373, W374, 5375, R376, A377, 5378, G379, P381, Q417, C418, R419, T421, P426, R427, A428 of a Cs3 domain and residues D278 and T281 of a Cs2 domain of human IgE of SEQ ID NO: 7 as explained above.

The antibodies of the invention may be assayed for specific binding by any method known in the art. Many different competitive binding assay format(s) can be used for epitope binding. The immunoassays which can be used include, but are not limited to, competitive assay systems using techniques such western blots, radioimmunoassays, ELISA, “sandwich” immunoassays, immunoprecipitation assays, precipitin assays, gel diffusion precipitin assays, immunoradiometric assays, fluorescent immunoassays, protein A immunoassays, and complement-fixation assays. Such assays are routine and well known in the art (see, e.g., Ausubel et al., eds, 1994 Current Protocols in Molecular Biology, Vol. 1, John Wiley & sons, Inc., New York).

C. Nucleic Acids, Vectors and Host Cells

Also provided herein are nucleic acid molecules comprising a nucleic acid sequence that encodes a recombinant immunoglobulin heavy chain of the invention. The recombinant nucleic acid may further comprise additional elements sufficient to direct expression of the recombinant immunoglobulin heavy chain in a mammalian cell, such as a human cell. The recombinant nucleic acid may be provided as an expression vector that allows expression of the recombinant protein in the cell. The vector may be a transient expression vector or a stable expression vector. Exemplary suitable expression vectors are known in the art. In some embodiments, the expression vector is a non-viral vector. In some embodiments the vector is a transposon. In some embodiments the expression vector is a viral vector, which is capable of integrating into the cellular genome to provide long-term, stable expression of an exogenous gene. Other suitable viral vectors include without limitation vectors derived from retrovirus, adenovirus, adeno-associated virus, lentivirus, herpes simplex virus, vaccinia virus, and pox virus. In some embodiments the vector is a lentiviral vector. In some embodiments the vector is a retroviral vector.

Also provided are recombinant host cells comprising a nucleic acid molecule that encodes a recombinant immunoglobulin heavy chain of the invention. The host cell may be a mammalian cell. The host cell may be a human cell. The host cell may be a HEK 293F cell.

Also provided herein are nucleic acid molecules and sets of nucleic acid molecules comprising nucleic acid sequences that encode a recombinant antibody of the invention. In some embodiments a single nucleic acid molecule may comprise all of the nucleic acid sequences that encode a recombinant antibody of the invention. In some embodiments a plurality of nucleic acid molecules may comprise all of the nucleic acid sequences that encode a recombinant antibody of the invention. For example, in an embodiment the immunoglobulin heavy chain of an antibody may be encoded by a first nucleic acid molecule and the immunoglobulin light chain of an antibody may be encoded by a second nucleic acid molecule. The recombinant nucleic acid or set of nucleic acids may each independently further comprise additional elements sufficient to direct expression of the recombinant antibody in a mammalian cell, such as a human cell. The recombinant nucleic acid or set of nucleic acids may be provided as an expression vector(s) that allows expression of the recombinant antibody in the cell. The vector(s) may be a transient expression vector(s) or a stable expression vector(s). Exemplary suitable expression vectors are known in the art. In some embodiments, the expression vector is a non-viral vector. In some embodiments the vector is a transposon. In some embodiments the expression vector is a viral vector, which is capable of integrating into the cellular genome to provide long-term, stable expression of an exogenous gene. Other suitable viral vectors include without limitation vectors derived from retrovirus, adenovirus, adeno-associated virus, lentivirus, herpes simplex virus, vaccinia virus, and pox virus. In some embodiments the vector is a lentiviral vector. In some embodiments the vector is a retroviral vector.

Also provided are recombinant host cells comprising a nucleic acid molecule or set of nucleic acid molecules that encode a recombinant antibody of the invention. The host cell may be a mammalian cell. The host cell may be a human cell. The host cell may be a HEK 293F cell.

D. Methods of Making Recombinant Antibodies

Methods of making recombinant antibodies of the invention are also provided. A recombinant host cell comprising a nucleic acid or set of nucleic acids encoding a recombinant antibody is cultured in suitable culture medium under conditions that allow expression of the immunoglobulin heavy chain and light chain of the antibody and secretion of antibodies into the culture medium. The culture medium may be collected and the antibody used in this form. Alternatively the antibody may be purified from the culture medium using any suitable technique, such as affinity chromatography. A composition comprising the purified antibody may then be provided.

E. Compositions

Also provided are pharmaceutical compositions comprising a recombinant antibody of the invention. Formulations of the recombinant antibody of the present invention may be prepared for storage by mixing said recombinant antibody having the desired degree of purity with optional pharmaceutically acceptable carriers, excipients or stabilizers (Remington's Pharmaceutical Sciences 16th edition, Osol, A. Ed., 1980), in the form of lyophilized formulations or aqueous solutions. Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN, PLURONICS or polyethylene glycol (PEG). The formulations to be used for in vivo administration may be sterilized. This is readily accomplished by filtration through sterile filtration membranes or other methods.

The recombinant antibody may also be formulated as immunoliposomes. A liposome is a small vesicle comprising various types of lipids, phospholipids and/or surfactant that is useful for delivery of a therapeutic agent to a mammal. Liposomes containing the recombinant antibody may be prepared by methods known in the art, such as described in Epstein et al., 1985, Proc Natl Acad Sci USA, 82:3688; Hwang et al., 1980, Proc Natl Acad Sci USA, 77:4030; U.S. Pat. Nos. 4,485,045; 4,544,545; and PCT WO 97/38731. Liposomes with enhanced circulation time are disclosed in U.S. Pat. No. 5,013,556. The components of the liposome are commonly arranged in a bilayer formation, similar to the lipid arrangement of biological membranes. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The recombinant antibody may also be entrapped in microcapsules prepared by methods including but not limited to coacervation techniques, interfacial polymerization (for example using hydroxymethylcellulose or gelatin-microcapsules, or poly-(methylmethacylate) microcapsules), colloidal drug delivery systems (for example, liposomes, albumin microspheres, microemulsions, nano-particles and nanocapsules), and macroemulsions. Such techniques are disclosed in Remington's Pharmaceutical Sciences 16th edition, Osol, A. Ed., 1980. Sustained-release preparations may be prepared. Suitable examples of sustained-release preparations include semipermeable matrices of solid hydrophobic polymer, which matrices are in the form of shaped articles, e.g. films, or microcapsules. Examples of sustained-release matrices include polyesters, hydrogels (for example poly(2-hydroxyethyl-methacrylate), or poly(vinylalcohol)), polylactides (U.S. Pat. No. 3,773,919), copolymers of L-glutamic acid and gamma ethyl-L-glutamate, non-degradable ethylene-vinyl acetate, degradable lactic acid-glycolic acid copolymers such as the LUPRON DEPOT.RTM. (which are injectable microspheres composed of lactic acid-glycolic acid copolymer and leuprolide acetate), poly-D-(−)-3-hydroxybutyric acid, and ProLease.RTM. (commercially available from Alkermes), which is a microsphere-based delivery system composed of the desired bioactive molecule incorporated into a matrix of poly-DL-lactide-co-glycolide (PLG).

Administration of the pharmaceutical composition comprising the recombinant antibody is preferably in the form of a sterile aqueous solution. This may be done in a variety of ways, including, but not limited to orally, subcutaneously, intravenously, intranasally, intraotically, transdermally, topically (e.g., gels, salves, lotions, creams, etc.), intraperitoneally, intramuscularly, intrapulmonary, vaginally, parenterally, rectally, or intraocularly. In some instances, the recombinant antibody may be directly applied as a solution or spray. As is known in the art, the pharmaceutical composition may be formulated accordingly depending upon the manner of introduction.

Subcutaneous administration may be preferable in some circumstances because the patient may self-administer the pharmaceutical composition.

As is known in the art, protein therapeutics are often delivered by IV infusion or bolus. The recombinant antibody of the present invention may also be delivered using such methods. For example, administration may venious be by intravenous infusion with 0.9% sodium chloride as an infusion vehicle.

The recombinant antibody may also be provided as a lyophilized powder and reconstituted prior to administration. For example, the lyophilized powder may be reconstituted with Sterile Water for Injection (SWFI) USP, prior to administration.

The concentration of the recombinant antibody in the formulation may vary from about 0.1 to 100 weight %. The concentration of the recombinant antibody in the formulation may vary from 0.003 to 1.0 molar.

F. Methods and Uses

In another aspect this invention provides methods of reducing an IgE-mediated allergic response in a subject, comprising administering a recombinant antibody of the invention to the subject. Because an IgE-mediated allergic response is a disease mechanism underlying many diseases, this invention also provides methods of preventing or treating an IgE-mediated disease in a subject, comprising administering a recombinant antibody of the invention to the subject. In some embodiments the IgE-mediated disease is selected from asthma and chronic idiopathic urticaria.

In a particular embodiement, the invention relates to a recombinant antibody according to the invention or a hybrid molecule according to the invention for use in the prevention or treatment of an IgE-mediated disease in a subject in need therof.

A therapeutically effective dose of the recombinant antibody may be administered. By “therapeutically effective dose” herein is meant a dose that produces the effects for which it is administered. The exact dose will depend on the objective, and will be ascertainable by one skilled in the art using known techniques. Dosages may range from 0.0001 to 100 mg/kg of body weight or greater, for example 0.1, 1, 10, or 50 mg/kg of body weight, such as 1 to 10 mg/kg, 1 to 3 mg/kg, 2 to 4 mg/kg, 3 to 6 mg/kg, 4 to 7 mg/kg, 5 to 8 mg/kg, 6 to 9 mg/kg, or 7 to 10 mg./kg. In some embodiments a total dosage of from 50 to 400 mg, 50 to 100 mg, 100 to 150 mg, 150 to 200 mg, 200 to 250 mg, 250 to 300 mg, 300 to 350 mg, or 350 mg to 400 mg is administered subcutaneously. In some embodiments a dose of 50 mg, 100 mg, 150 mg, 200 mg, 250 mg, 300 mg, 350 mg, or 400 mg is administered subcutaneously.

In some embodiments, only a single dose of the recombinant antibody is used. In other embodiments, multiple doses of the recombinant antibody are administered. The elapsed time between administrations may be 24 hours, 48 hours, 2-4 days, 4-6 days, 1 week, 2 weeks, or more than 2 weeks. The dose may be administered at a dosing interval of from one to six weeks, such as every week, every two weeks, every three weeks, every four weeks, every five weeks, or every six weeks.

In other embodiments the recombinant antibody of the present invention is administered in metronomic dosing regimes, either by continuous infusion or frequent administration without extended rest periods. Such metronomic administration may involve dosing at constant intervals without rest periods. Typically such regimens encompass chronic low-dose or continuous infusion for an extended period of time, for example 1-2 days, 1-2 weeks, 1-2 months, or up to 6 months or more. The use of lower doses may minimize side effects and the need for rest periods.

In certain embodiments a clinically relevant reduction in an IgE-mediated allergic response in the subject is observed following administration of the recombinant antibody. In some embodiments the clinically relevant reduction in an IgE-mediated allergic response is a reduction that is at least 20%, 30%, 40%, 50%, 60%, 70%, 80%, 90%, 95%, or 100% of the reduction in the IgE-mediated allergic response observed following administration of omalizumab.

In certain embodiments the recombinant antibody is administered at a dose and a dosing interval that results in severe systemic allergic shock in fewer than 0.2% of patients receiving the same dose and dosing interval in a controlled clinical study of similar patients. In certain embodiments the recombinant antibody is administered at a dose and a dosing interval that results in severe systemic allergic shock in fewer than 0.1% of patients receiving the same dose and dosing interval in a controlled clinical study of similar patients. In certain embodiments the recombinant antibody is administered at a dose and a dosing interval that results in severe systemic allergic shock in fewer than 0.05% of patients receiving the same dose and dosing interval in a controlled clinical study of similar patients. In certain embodiments the recombinant antibody is administered at a dose and a dosing interval that results in severe systemic allergic shock in fewer than 0.01% of patients receiving the same dose and dosing interval in a controlled clinical study of similar patients.

In certain embodiments the recombinant antibody is administered at a dose and a dosing interval that results in skin inflammation in fewer than 40% of patients receiving the same dose and dosing interval in a controlled clinical study of similar patients. In certain embodiments the recombinant antibody is administered at a dose and a dosing interval that results in severe systemic allergic shock in fewer than 20% of patients receiving the same dose and dosing interval in a controlled clinical study of similar patients. In certain embodiments the recombinant antibody is administered at a dose and a dosing interval that results in severe systemic allergic shock in fewer than 10% of patients receiving the same dose and dosing interval in a controlled clinical study of similar patients. In certain embodiments the recombinant antibody is administered at a dose and a dosing interval that results in severe systemic allergic shock in fewer than 5% of patients receiving the same dose and dosing interval in a controlled clinical study of similar patients.

Also provided herein are compositions for use in reducing an IgE-mediated allergic response in a subject. The compositions may comprise a recombinant antibody of the invention. Because an IgE-mediated allergic response is a disease mechanism underlying many diseases, this invention also provides compositions for use in preventing or treating an IgE-mediated disease in a subject. The compositions may comprise a recombinant antibody of the invention. In some embodiments the IgE-mediated disease is selected from asthma and chronic idiopathic urticaria.

Also provided herein is the use of compositions comprising a recombinant antibody of the invention for reducing an IgE-mediated allergic response in a subject. Because an IgE-mediated allergic response is a disease mechanism underlying many diseases, this invention also provides use of compositions comprising a recombinant antibody of the invention for preventing or treating an IgE-mediated disease in a subject. In some embodiments the IgE-mediated disease is selected from asthma and chronic idiopathic urticaria.

Throughout the specification, several terms are employed and are defined in the following paragraphs.

The invention will be further illustrated by the following figures and examples. However, these examples and figures should not be interpreted in any way as limiting the scope of the present invention.

### FIGURES

FIG. 1. Immune complexes (ICs) made with Omalizumab and human IgE can bind human FcγRs, and Omalizumab binds human C1q. A. We assessed binding of preformed ICs made of Omalizumab and human IgE to FcγRs using a bank of CHO cells stably transfected with each one of the human FcγRs (Bruhns et al., Blood 2009 Apr. 16; 113(16):3716-25). Our results show that ICs formed with Omalizumab and human IgE can bind all activating FcγRs (I, IIA, IIIA & IIIB), but not the inhibitory FcγRIIB B. Plate-bound Omalizumab binds human C1q in a dose dependent manner. Plates were coated with increasing doses of Omalizumab, followed by incubation with human C1q and detection with an anti-human C1q antibody. Anti-mouse platelets mAb clone 6A6 expressed as a human IgG1 isotype and mutated in its Fc portion at position 322 (K322A) to preclude binding to C1q was used as a negative control.

FIG. 2. ICs made of Omalizumab and human IgE can activate neutrophils through engagement of human FcγRs. A. Representative flow cytometry analysis of human neutrophils purified from the blood of healthy donors. Data are gated on live CD45+ cells and neutrophils are defined as CD45+CD15+CD66b+ cells. B. Percentage of CD62L^{high} human neutrophils (CD45+CD15+CD66b+) after incubation with IgE: Omalizumab immune complexes (ICs) or, as a control, human IgE alone or medium alone. C. Representative flow cytometry analysis of neutrophils purified from the bone marrow of ‘humanized’ hFcγR^{KI} mice (Beutier H, et al. Science Immunology, in press. Apr. 13, 2018) or FcγR^{null} mice (Gillis C M, et al. J Autoimmun 2016). Data are gated on live CD45+ cells and neutrophils are defined as CD45+CD11b+Ly6G+ cells. D. CD62L expression on CD11b+Ly6G+ neutrophils purified form hFcγR^{KI} or FcγR^{null} mice after incubation with IgE:Omalizumab immune complexes (ICs) or, as a control, medium alone.

FIG. 3. ICs made of Omalizumab and human IgE induce anaphylaxis through engagement of hFcγRs in humanized mice. Preformed Omalizumab:IgE ICs were injected i.v. into ‘humanized’ hFcγR^{KI} mice (Beutier H, et al. Science Immunology, in press. Apr. 13, 2018) or FcγR^{null} mice (n=9 per group) (Gillis C M, et al. J Autoimmun 2016). As a control, groups of hFcγR^{KI} mice were injected with human IgE alone or Omalizumab alone (n=6 per group). Development of anaphylaxis was monitored during 1 h by measuring changes in body temperature (anaphylaxis induces hypothermia in mice).

FIG. 4. Fc-engineered anti-IgE mAbs have reduced binding to FcγRs and C1q and do not activate neutrophils. We cloned and produced WT or Fc-engineered anti-IgE mAbs carrying L234A/L235A or N297A mutations in the Fc portion (referred to as ‘LALA’ and ‘NA’ anti-IgE mAbs, respectively) using Omalizumab VH and VL sequences. A. Binding of preformed ICs made of human IgE and WT or Fc-engineered ‘LALA’ or ‘NA’ anti-IgE mAbs to FcγRs using a bank of CHO cells stably transfected with each one of the human FcγRs (Bruhns et al., Blood 2009 Apr. 16; 113(16):3716-25). B. Binding of WT or Fc-engineered ‘LALA’ or ‘NA’ anti-IgE mAbs to human C1q assessed by ELISA. C. Percentage of CD62L^{high} cells after in vitro incubation of purified human neutrophils with ICs made of human IgE and WT or Fc-engineered ‘LALA’ or ‘NA’ anti-IgE mAbs, or IgE alone as a control.

FIG. 5. WT and Fc-engineered anti-IgE mAbs have similar half-life in vivo. We injected 100 μg of WT or Fc-engineered ‘NA’ anti-IgE mAbs intraperitoneally (i.p.) into hFcγR^{KI} hFcRn^{KI} mice (unpublished), and collected serum at different time-points. Levels of anti-IgE mAbs were measured by ELISA. Estimated half-life of each mAb is indicated.

FIG. 6. Fc-engineered anti-IgE mAbs are as potent as Omalizumab at blocking IgE. A. Binding of Omalizumab and the WT or Fc-engineered anti-IgE mAbs to human IgE assessed by ELISA. B. WT and Fc-engineered anti-IgE mAbs can equally block binding of human IgE and subsequent IgE-mediated degranulation in human mast cells in vitro. Mast cells were pre-treated with the indicated anti-IgE mAb, then sensitized with anti-nitrophenyl (NP) human IgE. 16 h later, cells were stimulated with NP to induce degranulation. Mast cell degranulation was assessed here by flow cytometry by measuring levels of fluorescent avidin which binds to heparin contained in mast cell granules and exposed to mast cell surface upon degranulation. C. hFcεRI^{Tg} mice (Dombrowicz D, et al. J Immunol 1996; 157:1645-51), which express the human IgE receptor, were injected intravenously (i.v.) with Omalizumab or the N297A (‘NA’) mutant anti-IgE mAb (700 μg), or PBS as a control. 30 min later, mice were sensitized i.v. with anti-NP human IgE (10 μg). 16 h later, mice were challenged with 500 μg NP coupled to BSA (NP-BSA) to induce anaphylaxis. Anaphylaxis was monitored during 1 h by measuring changes in body temperature (anaphylaxis induces hypothermia in mice). We used hFcεRI⁻ mice (Dombrowicz D, et al. Cell 1993; 75:969-76) (which do not respond to human IgE) as a control.

FIG. 7. Fc-engineered anti-IgE mAbs do not induce anaphylaxis in humanized hFcγR^{KI} mice. We compared the ability of ICs made of IgE and Omalizumab or IgE and the Fc-engineered ‘NA’ anti-IgE mAb to induce anaphylaxis in hFcγR^{KI} mice (Beutier H, et al. Science Immunology, in press. Apr. 13, 2018). Preformed ICs were injected i.v. into hFcγR^{KI} mice and anaphylaxis was monitored during 1 h by measuring changes in body temperature (anaphylaxis induces hypothermia in mice).

### EXAMPLE

Material & Methods and Results

### Example 1

Mice. hFcγR^{KI} mice (Beutier et al. Science Immunol, in press) were generated by Regeneron Pharmaceuticals, Inc. on a mixed 62.5% C57BL/6N, 37.5% 12956/SvEv genetic background, and backcrossed one generation to C57BL/6N. FcγR^{null} mice Gillis C M, et al. J Autoimmun 2016) and hFcεRI^{Tg} mice (Dombrowicz et al. J Immunol 1996) were described previously. hFcγR^{KI} hFcRn^{KI} mice were generated by intercrossing of hFcγR^{KI} mice with hFcRn^{KI} mice (VG1481) designed and generated by Regeneron Pharmaceuticals, Inc. nude-hFcγR^{KI} and nude-hFcR^{null} mice were obtained by intercros sing NMRI-Foxn1^{nu/nu} (Nude) (Janvier labs) mice with hFcγR^{KI} and FcγR^{null} mice, respectively. All mice were bred at Institut Pasteur and demonstrated normal development and breeding patterns. We used age-matched mice for all experiments. All animal care and experimentation were conducted in compliance with the guidelines and specific approval of the Animal Ethics committee CETEA (Institut Pasteur, Paris, France) registered under #2013-0103, and by the French Ministry of Research under agreement #00513.02.

Production of human IgE antibodies. JW8/5/13 (ECACC 87080706) cells were obtained from Sigma-Aldrich. This cell line produces a chimeric human IgE antibody directed against the hapten 4-hydroxy-3-nitrophenacetyl (NP), and composed of the human Fc c chain and mouse anti-NP variable chain (we refer to this antibody as ‘human IgE’ in the manuscript). JW8/5/13 cells were cultured in complete Dulbeco modified Eagle medium (DMEM, Gibco) containing 2 mM glutamine and 10% Foetal Bovine Serum (FBS) at 9×10⁵ cells/ml. After 15 days, supernatants were harvested, centrifuged at 4200 rpm for 30 min and filtered (0.2 μm). We purified IgE antibodies by affinity chromatography. Briefly, CNBr-activated Sepharose 4 Fast Flow Beads (GE Healthcare) were coupled with omalizumab using a ratio of 2.5 mg of protein for each gram of beads. Beads were weighted, washed with 15 volumes of cold 1 mM HCl and centrifuged for 5 min at 2500 rpm. Omalizumab was resuspended in coupling solution (0.1 M NaHCO₃ pH 8.3 containing 0.5M NaCl) and mixed with beads overnight at 4° C. under agitation. Beads were washed with coupling buffer and non-reacted groups were blocked with 0.1 M Tris-HCl buffer pH 8.0. Omalizumab-coupled beads were then washed using alternate low (0.1 M acetate buffer pH 3) and high (0.1 M Tris-HCl pH 8) pH solutions and stored in Borate buffer (100 mM Borate, 150 mM NaCl pH 8.0) at 4° C. until use. For purification of IgE, Omalizumab-coupled sepharose beads were packed in XK 16/20 Column (GE Healthcare) and affinity chromatography was performed using an AKTA pure FPLC instrument (GE Healthcare). After purification, IgE antibodies were desalted with HiTrap Desalting Column (GE Healthcare), and stored at 4° C. until use. For some experiments, purified IgE antibodies were conjugated with FITC using the Pierce Antibody labelling kit (Thermo Fisher Scientific) according to the manufacturer's instructions.

Cloning and production of WT and Fc-engineered anti-IgE Abs. We obtained Omalizumab V_{H} and V_{L} sequences from a publicly available website (https://www.drugbank.ca/drugs/DB00043):

Omalizumab V_{H} (SEQ ID NO: 1):
EVQLVESGGGLVQPGGSLRLSCAVSGYSITSGYSWNWIRQAPGKGLEWVA
SITYDGSTNYADSVKGRFTISRDDSKNTFYLQMNSLRAEDTAVYYCARGS
HYFGHWHFAVWGQGTLVTVSS
Omalizumab V_{L} (SEQ ID NO: 2):
DIQLTQSPSSLSASVGDRVTITCRASQSVDYDGDSYMNWYQQKPGKAPKL
LIYAASYLESGVPSRFSGSGSGTDFTLTISSLQPEDFATYYCQQSHEDPY
TFGQGTKVEIK

Omalizumab V_{H} and V_{L} sequences were reverse transcribed into DNA. V_{H} and V_{L} DNA fragments were synthesized by Eurofins. Omalizumab V H sequence was cloned into a human pUC19-Igγ1-expressing vector (a kind gift from Hugo Mouquet, Institut Pasteur, Paris) using SalI and AgeI restriction sites, and Omalizumab V L sequence was cloned into human Igκ-expressing vector using AgeI and BsiWI restriction sites, as previously described (Tiller et al, J Immunol Methods 2008). For Fc-engineered mAbs, point mutations in the Igγ1-expressing vector were introduced at position 297 (N297A, thereafter named ‘NA’ mutant) and 234/235 (L234A/L235A, thereafter named ‘LALA’ mutant) using the QuickChange Site-Directed Mutagenesis Kit (Agilent Technologies), according to the manufacturer's instructions. All vectors were sequenced before being used for antibody production.

WT anti-IgE mAb heavy chain (SEQ ID NO: 3):
EVQLVESGGGLVQPGGSLRLSCAVSGYSITSGYSWNWIRQAPGKGLEWVA
SITYDGSTNYADSVKGRFTISRDDSKNTFYLQMNSLRAEDTAVYYCARGS
HYFGHWHFAVWGQGTLVTVSSGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
Fc-engineered ‘LALA’ anti-IgE mAb heavy chain
(SEQ ID NO: 4):
EVQLVESGGGLVQPGGSLRLSCAVSGYSITSGYSWNWIRQAPGKGLEWVA
SITYDGSTNYADSVKGRFTISRDDSKNTFYLQMNSLRAEDTAVYYCARGS
HYFGHWHFAVWGQGTLVTVSSGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKRVEPKSCDKTHTCPPCPAPEAAGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
Fc-engineered ‘NA’ anti-IgE mAb heavy chain
(SEQ ID NO: 5):
VQLVESGGGLVQPGGSLRLSCAVSGYSITSGYSWNWIRQAPGKGLEWVAS
ITYDGSTNYADSVKGRFTISRDDSKNTFYLQMNSLRAEDTAVYYCARGSH
YFGHWHFAVWGQGTLVTVSSGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTCPPCPAPELLGGPSVFLFPPKPKDTL
MISRTPEVTCVVVDVSHEDPEVKFNWYVDGVEVHNAKTKPREEQYASTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK
Light chain common to the WT, LALA and NA
anti-IgE mAbs (SEQ ID NO: 6):
DIQLTQSPSSLSASVGDRVTITCRASQSVDYDGDSYMNWYQQKPGKAPKL
LIYAASYLESGVPSRFSGSGSGTDFTLTISSLQPEDFATYYCQQSHEDPY
TFGQGTKVEIKRTVAAPSVFIFPPSDEQLKSGTASVVCLLNNFYPREAKV
QWKVDNALQSGNSQESVTEQDSKDSTYSLS STLTLSKADYEKHKVYACE
VTHQGLSSPVTKSFNR

Antibodies were produced by transient co-transfection of one of the heavy chain expression plasmids (WT, LALA, or NA) and the light chain expression plasmid into exponentially growing HEK 293F cells. Freestyle™ 293-F suspension cells were cultured in serum-free Freestyle™ 293 Expression Medium (Life Technologies). 293-F cells were grown in suspension at 37° C. in a humidified 8% CO₂ incubator on a shaker platform rotating at 110 rpm. Twenty-four hours before transfection, cell were harvested by centrifugation at 300 g for 5 min, and resuspended in Freestyle™ 293 expression medium at a density of 1×10⁶ cells/ml, and cultured overnight in the same conditions as mentioned above. For the production of mAbs, 40 μg of V_{H} and V_{L} expressing plasmids were diluted in 80 μl of FectoPRO reagent (PolyPlus) at a final DNA concentration of 0.8 μg/ml, incubated for 10 minutes at RT before addition to the cells. Twenty-four hours post-transfection, cells were diluted 1:1 with Freestyle™ 293 expression medium. Cells were cultured for 6 days after transfection, supernatants were harvested, centrifuged at 4200 rpm for 30 min and filtered (0.2 μm). Antibodies were purified by affinity chromatography using an AKTA pure FPLC instrument (GE Healthcare) and HiTrap Protein G Column (GE Healthcare). After purification, mAbs were desalted with HiTrap Desalting Column (GE Healthcare).

In vitro formation of Omalizumab-IgE immune complexes (ICs). All antibodies were centrifuged at 13.000 g for 15 min to remove any possible aggregate in stock solution. ICs were formed by incubating anti-NP human IgE or FITC-labelled anti-NP human IgE with omalizumab or Fc-engineered anti-IgE mAbs at a 1:2 ratio for 1 h at 37° C. under agitation (250 rpm).

Binding of ICs to FcγRs expressed on CHO cells. We use a collection of Chinese Hamster Ovarian (CHO) transfectants expressing FLAG-tagged human FcγRs (Bruhns et al. Blood 2009) to assess binding of preformed ICs to various FcγRs. Briefly, preformed ICs made of FITC-labelled IgE and omalizumab or Fc-engineered anti-IgE mAbs were incubated with 5×10⁴ CHO transfectants for 1 h on ice. Cells were washed with MACS buffer (PBS/0.5% BSA/2 mM EDTA). Cell bound ICs were detected using MACSQuant flow cytometer (Miltenyi Biotec), and data were analyzed using Flowjo Software (Tree Star). CHO transfectants incubated with FITC-labelled IgE alone were used as a negative control. Expression of human FcγR on the surface of each CHO transfectant was confirmed by flow cytometry using antibodies against FcγRIA (10.1), FcγRIIA/IIB (AT.10) and FcγRIIIA/IIIB (MEM-154), all from BD Biosciences.

Binding of Omalizumab or Fc-engineered anti-IgE mAbs to human C1q. To measure binding of Omalizumab or Fc-engineered anti-IgE mAbs to human C1q, 96-well plates (Costar) were coated with increasing concentrations of each mAb (12.5 to 200 ng/well) in 50 nM carbonate-bicarbonate buffer (pH 9.6) at 4° C. for 16 h. Plates were washed 3 times with PBS containing 0.05% Tween 20 (PBST), and blocked for 2 h at room temperature in PBST contaning 0.1% gelatine and 3% BSA. Plates were washed 3 times before addition of native human C1q (Abd Serotec) at 1 ng/μL. After 16 h, plates were washed with PB ST and incubated 1 h with 50 μL of PBS containing 1 μg/mL-anti-human C1q HRP (Abd Serotec). Plates were washed 3 times with PBST before addition of 100 μL/well OPD peroxidase (Sigma). Reaction was stopped by addition of 50 μL 2M H₂SO₄ and absorbance was recorded at 492 nm and corrected at 620 nm.

Binding of Omalizumab or Fc-engineered anti-IgE mAbs to human IgE. To measure binding of Omalizumab or Fc-engineered anti-IgE mAbs to human IgE, 96-well plates (Costar) were coated with each mAb (0.5 μg/well) in 50 nM carbonate-bicarbonate buffer (pH 9.6) at 4° C. for 16 h. Plates were washed 3 times with PBST, and blocked for 2 h at room temperature in PBST 1% BSA. Plates were washed 3 times before addition of increasing doses of IgE (1.6 to 5000 ng/well). After 3 hours, plates were washed with PBST and incubated with 1:10.000 of anti-human IgE (Bethyl) for 1 h. Plates were washed 3 times with PBST before addition of 100 μL/well OPD peroxidase (Sigma). Reaction was stopped by addition of 50 μL 2M H₂SO₄ and absorbance was recorded at 492 nm and corrected at 620 nm.

Recirculation of Fc-engineered anti-IgE antibodies in vivo. hFcγR^{KI} hFcRn^{KI} mice were injected i.p. with 100 μg of WT or Fc-engineered anti-IgE mAbs in 100 μL 0.9% NaCl solution. Serum was then collected every 7 or 14 days starting from day 1 post-injection and stored at −20° C. until use. Serum levels of anti-IgE mAbs were quantified by ELISA. Briefly, 96-well plates (Costar) were coated with F(ab′)2 Goat Anti-human IgG (5 μg/mL; Jackson ImmunoResearch) in 50 nM carbonate-bicarbonate buffer (pH 9.6) at 4° C. for 16 h. Plates were washed 3 times with PBST, and blocked for 2 h at room temperature in PBST containing 1% BSA. Plates were washed 3 times before addition of serial dilutions of serum (1/100 to 1/3000). After 3 hours, plates were washed with PBST and incubated with goat anti-human kappa HRP (1:4.000; Southern Biotech) for 1 h. Plates were washed 3 times with PBST before addition of 100 μL/well OPD peroxidase (Sigma). Reaction was stopped by addition of 50 μL 2M H₂SO₄ and absorbance was recorded at 492 nm and corrected at 620 nm.

IC-mediated activation of neutrophils. Human EDTA-collected blood from healthy donors was obtained from the blood bank (Établissement Francais du Sang EFS). Human neutrophils were purified with MACSxpress Neutrophil Isolation Kit (Miltenyi) according to the manufacturer's instructions, and neutrophils purity was assessed by flow cytometry (human neutrophils were defined as CD45⁺ CD15⁺CD66⁺ cells). Purified human neutrophils were primed in RPMI medium containing 10% FCS, 10 ng/ml clinical grade G-CSF (Miltenyi) and 50 ng/ml recombinant human IFN-γ (Miltenyi) at 5×10⁵ cells/ml for 16 h before activation with ICs. Mouse neutrophils were purified from the tibia and femur of hFcγR^{KI} and hFcγR^{null} mice by negative selection using the EasySep Mouse Neutrophil Enrichment kit (STEMCELL Technologies; >90% Ly6G⁺ CD11b⁺ on average) according to the manufacturer's instructions, and neutrophils purity was assessed by flow cytometry (mouse neutrophils were defined as CD45⁺CD11b⁺ Ly6G⁺ cells). Purified mouse neutrophils were primed in RPMI medium containing 10% FCS, 10 ng/ml mouse M-CSF (Miltenyi) and 50 ng/ml mouse IFN-γ (Miltenyi) at 5×10⁵ cells/ml for 16 h before activation with ICs. Activation of human or neutrophils by ICs was performed as previously described (Jakus et al. J Immunol 2008). Briefly, immobilized ICs were formed by coating 96-well plates (Costar) with human IgE (50 μg/ml) in 50 mM carbonate/bicarbonate buffer (pH 9.6) for 16 h, followed by blocking with 10% Ultra Low IgG FBS (Invitrogen) in PBS for 2 h and an incubation with Omalizumab or Fc-engineered anti-IgE mAbs at 100 μg/ml for 1 h in PBST. Plates were washed 3 times with PBS, and purified neutrophils were incubated at 5×10⁴ cells/well for 1 h at 37° C. Human neutrophils were stained with fluorescently-labelled anti-CD45, anti-CD15, anti-CD66 and anti-CD62L antibodies for 30 min at 4° C. Mouse neutrophils were stained with fluorescently-labelled anti-CD45, anti-CD11b, anti-Ly6G and anti-CD62L antibodies for 30 min at 4° C. Activation of mouse or human neutrophils was assessed by measuring changes in expression of CD62L. Data were acquired using MACSQuant flow cytometer (Miltenyi), and analyzed with Flowjo Software (Tree Star). Dead cells (identified by staining with propidium iodide; Gibco) were not included in the analysis.

ICs-mediated passive systemic anaphylaxis (PSA). Pre-formed ICs were diluted in saline and injected i.v. in hFcγR^{KI} or FcγR^{null} mice at a final concentration of 250 vg IgE and 500 μg anti-IgE in 100 μL to induce anaphylaxis. We injected saline, IgE or anti-IgE alone (diluted in the same conditions) as a control. Rectal temperature measurements were performed using a digital thermometer (YSI) immediately before (time 0) and at different time points for up to 1 h after injection of ICs.

Human IgE-mediated passive systemic anaphylaxis (PSA). hFcεRI^{Tg} mice were injected intravenously (i.v.) with 700 vg anti-IgE IgG1 (Omalizumab or Fc-engineered anti-IgE mAbs) in 100 μL saline, or saline only as a control. 30 min later, mice were sensitized i.v. with 10 vg anti-NP human IgE. 16 h later, mice were challenged i.v. with 500 μg NP-BSA (Santa Cruz). Rectal temperature measurements were performed using a digital thermometer (YSI) immediately before (time 0) and at different time points for up to 1 h after challenge with NP.

Generation of peripheral blood derived-cultured human mast cells (hMCs) and degranulation analysis. Peripheral blood mononuclear cells were separated using Ficoll-Paque PLUS (GE Healthcare) and CD34⁺ cells were isolated with a Human CD34 Positive Selection Kit (StemCell Technologies). Cells were seeded at 1×10⁶ cells/mL in StemSpan medium (StemCell Technologies) supplemented with Ciprofloxacin (10 vg/ml; Sigma-Aldrich), recombinant human IL-6 (50 ng/ml; Peprotech), human IL-3 (50 ng/ml; Peprotech) and SCF (100 ng/mL; Miltenyi). Every three to four days, cultures were doubled in volume with fresh supplemented medium for 30 days. Cells were then progressively transferred to Iscove's modified Dulbeccos medium (IMDM; Gibco) supplemented with 50 μM 2-mercaptoethanol (Life Technologies), 0.5% BSA, 1% Insulin-Transferrin-Selenium (Life Technologies), Ciprofloxacin (10 ug/ml), human IL-6 (50 ng/mL) and human SCF (100 ng/mL). hMC were supplemented with fresh medium every week. All data presented were generated with cells after weeks of culture, and co-expression of FcεRI and CD117 was verified by flow cytometry. For IgE-mediated degranulation experiments, 5×10⁴ hMCs were plated in 50 μl of supplemented IMDM medium and incubated with increasing doses of Omalizumab or Fc-engineered mAbs. 2 μg/ml FITC-labelled human IgE were added to the culture immediately after. 16 h later, hMCs were washed and stimulated with NP-BSA at 37° C. Cells were washed after 1 h and incubated with FITC-coupled avidin (BD Biosciences). Degranulation was assessed by quantifying the percentage of FITC⁺ hMCs by flow cytometry. Data were acquired using MACSQuant flow cytometer (Miltenyi Biotec), and analyzed with Flowjo Software (Tree Star).

Statistical analyses. Data are presented as mean±SEM. Temperature loss during PSA was compared by using 2-way repeated-measures ANOVA. Experiments with human neutrophils were analysed using one-way analysis of variance (ANOVA) with Tukey's post test. Statistical analyses were performed with Prism Software (GraphPad Software, La Jolla, Calif). P values <0.05 are considered statistically significant.

### Example 2

Omalizumab-IgE Immune Complexes (ICs) Bind Human FcγRs and Complement C1q

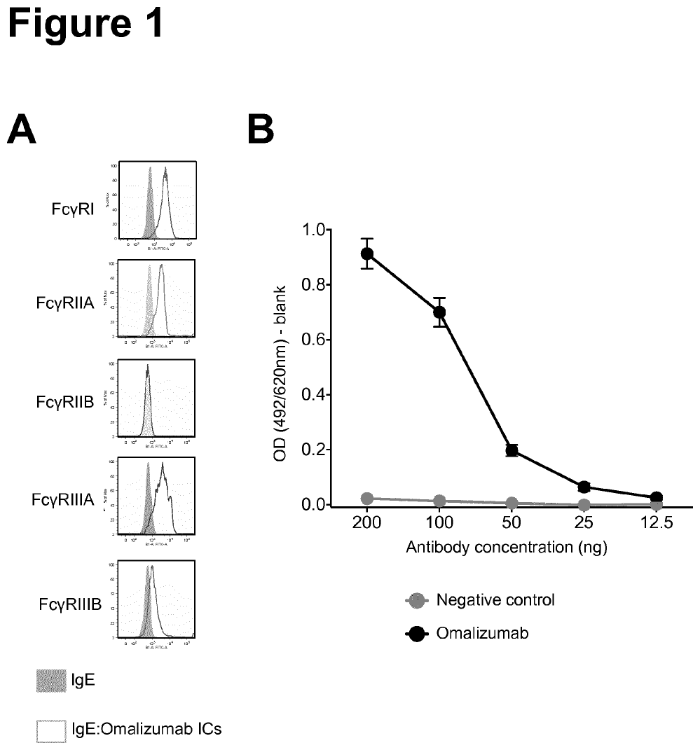

We first incubated FITC-labelled human IgE with Omalizumab to form immune complexes (ICs) in vitro. We then incubated such ICs, or FITC-labelled IgE as a negative control, with CHO transfectants expressing each one of the human IgG FcγR. As expected, IgE alone did not bind FcγRs (FIG. 1A). By contrast, ICs made of IgE and Omalizumab bound all activating FcγRs (FcγRI, IIA, IIIA and IIIB) but not the inhibitory FcγRIIB (FIG. 1A). We then assess binding of Omalizumab to the C1q component of complement by ELISA, and found that plate-bound Omalizumab can bind human C1q in a dose dependent manner (FIG. 1B). Thus, our results show that Omalizumab-IgE ICs have the potential to induce inflammation through engagement of human FcγRs or activation of the human complement cascade.

### Example 3

Omalizumab-IgE ICs Activate Neutrophils Through FcγRs

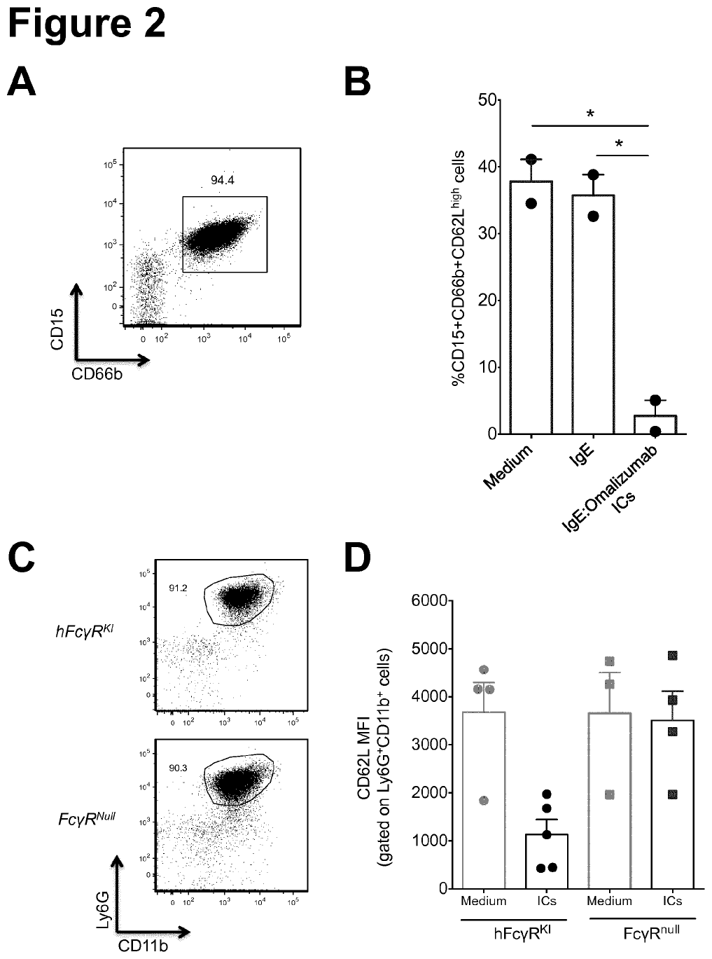

We next assessed whether Omalizumab-IgE ICs can activate neutrophils through engagement of FcγRs. We purified human neutrophils from healthy donors (FIG. 2A) and incubated these cells with plate-bound Omalizumab-IgE ICs. We found that such ICs induce marked downregulation of CD62L on the surface of neutrophils, a hallmark of neutrophil activation (FIG. 2B). We perform similar experiments with neutrophils purified from hFcγR^{KI} mice (in which all mouse FcγR have been replaced with the human FcγRs (Gillis et al., J Allergy Clin Immunol 2017; Beutier et al. Sci Immunol, 2018) or FcγR^{null} mice (Gillis C M, et al. J Autoimmun 2016) as a control (FIG. 2C). We observed that Omalizumab-IgE ICs induced a downregulation of CD62L in neutrophils purified from hFcγR^{KI} mice but not in neutrophils purified from FcγR^{null} mice (FIG. 2D). Altogether, these results indicate that Omalizumab-IgE can activate neutrophils through engagement of human FcγRs.

### Example 4

Omalizumab-IgE ICs Induce Local Skin Inflammation and Anaphylaxis Through FcγRs in Humanized hFcγRKI Mice

The most frequent side effect observed with Omalizumab is skin inflammation at the site of subcutaneous injection of the drug. We hypothesized that such local inflammation could be a consequence of active FcγR engagement. To assess this in vivo, we injected Omalizumab/IgE ICs subcutaneously into nude hFcγR^{KI} mice and nude FcγR^{null} mice, and assessed skin inflammation 2 h after injection by bioluminescence imaging of myeloperoxidase (MPO) activity after luminol administration. We observed a strong MPO activity at the site of Omalizumab/IgE ICs injection in hFcγR^{KI} mice (data not shown). By contrast, MPO activity was markedly reduced at the site of injection of IgE alone or Omalizumab alone, and at the site of injection of ICs in FcγR^{null} mice. Thus, our results indicate that Omalizumab/IgE ICs can induce local skin inflammation through engagement of human FcγRs.

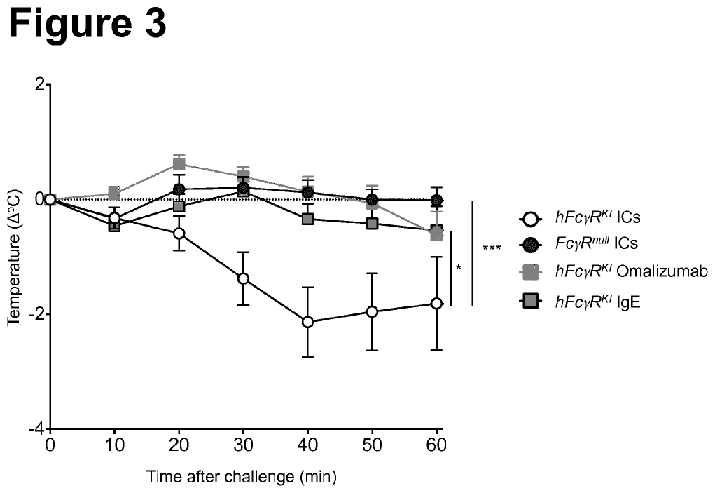

The most dramatic side effect reported for Omalizumab is systemic anaphylaxis. We thus assessed whether Omalizumab-IgE can induce anaphylaxis in hFcγR^{KI} mice. Intravenous injection of Omalizumab-IgE ICs, but not of IgE or Omalizumab alone, induced significant hypothermia (the main readout of anaphylaxis in mice) in hFcγR^{KI} mice (FIG. 3). Importantly, hypothermia was not observed in hFcγR^{null} mice (FIG. 3), demonstrating that Omalizumab-IgE ICs induce systemic anaphylaxis through engagement of human FcγRs.

Since these results were obtained with injection of preformed ICs, we next assessed whether in vivo formation of Omalizumab/IgE ICs could trigger anaphylaxis in hFcγR^{KI} mice. However, injection of human IgE followed by injection of Omalizumab in naïve hFcγR^{KI} mice did not induce signs of anaphylaxis (data not shown). Anaphylaxis to Omalizumab remains a rare event, occurring in 0.1-0.2% of Omalizumab-treated patients. Interestingly, three clinical studies have shown that occurrence of anaphylaxis to Omalizumab is significantly increased in patients with prior history of anaphylaxis unrelated to Omalizumab. It is thus possible that a subpopulation of highly atopic patients is more prone to develop anaphylaxis to Omalizumab. To mimic this, we pre-treated hFcγR^{KI} mice with IL-4C, a long-lasting formulation of IL-4. Strikingly, IL-4C-treated mice developed marked anaphylaxis upon sequential injection of human IgE and Omalizumab (data not shown). Altogether, our data in humanized mice are consistent with prior clinical observations which suggest that the risk of anaphylaxis to Omalizumab could be markedly increased in highly atopic subjects.

### Example 5

Fc-Engineered Anti-IgE mAbs have Reduced Binding to FcγRs and C1q and do not Activate Neutrophils

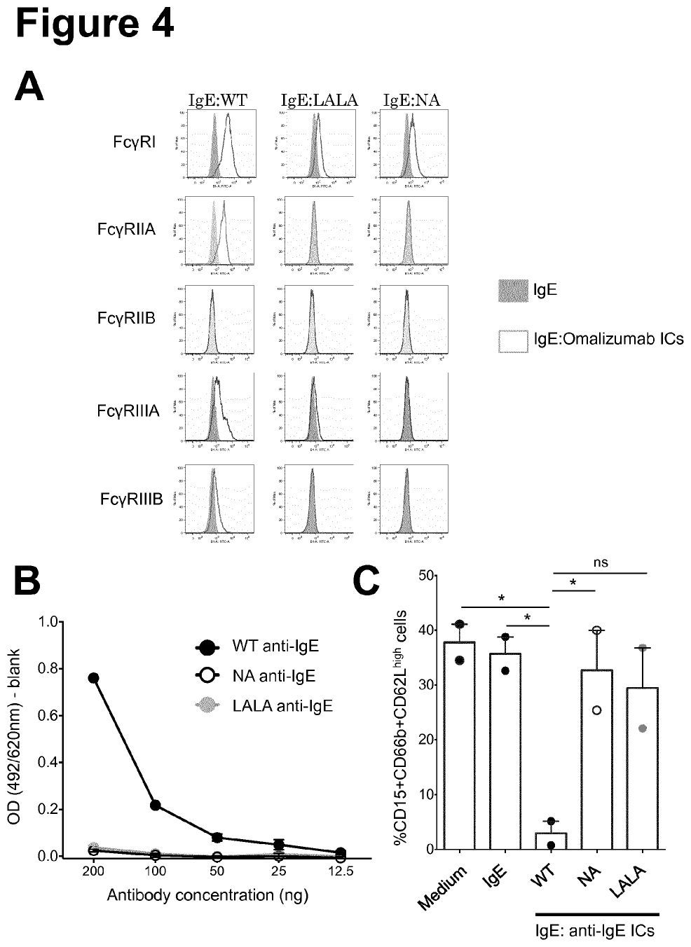

Based on these results, we decided to clone and produce Fc-engineered forms of Omalizumab in which the Fc portion of this IgG1 mAb is mutated to reduce binding to FcγRs and complement. We produced two Fc-engineered anti-IgE IgG1 mAb (using Omalizumab VH and VL sequences) with a L234A/L235A or a N297A mutation in the Fc portion (these Fc-engineered mAbs are referred to as ‘LALA’ and ‘NA’ anti-IgE mAbs). As a control, we also produced a WT anti-IgE IgG1 mAb. As expected, ICs made of human IgE and the WT anti-IgE mAb could bind all activating human FcγRs (FIG. 4A), similarly to what we observed when using ICs made of IgE and Omalizumab (FIG. 1A). However, binding to FcγRs was markedly reduced with ICs made of human IgE and the Fc-engineered ‘LALA’ or ‘NA’ anti-IgE mAbs (FIG. 4A). Plate-bound WT anti-IgE mAbs could bind human C1q (FIG. 4B), as observed with commercial Omalizumab (FIG. 1B). By great contrast, we observed no binding to C1q to both Fc-engineered ‘LALA’ and ‘NA’ anti-IgE mAbs (FIG. 4B). Finally, we observed activation of human neutrophils with plate-bound ICs made of IgE and WT anti-IgE mAbs, but not with ICs made of IgE and Fc-engineered ‘LALA’ or ‘NA’ anti-IgE mAbs (FIG. 4C). Altogether, these results show that the Fc-engineered anti-IgE mAbs we produced have markedly reduced binding to FcγRs and complement, and do not activate neutrophils.

### Example 6

WT and Fc-Engineered Anti-IgE mAbs have Similar Half-Life In Vivo

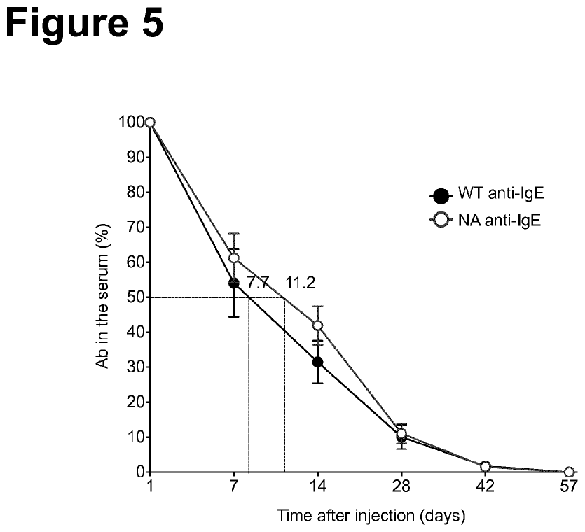

Our results showed that both Fc-engineered “LALA” or “NA” anti-IgE mAbs perform equally in the in vitro experiments. So, to limit the number of animals we decided to perform in vivo experiments with the Fc-engineered ‘NA’ anti-IgE mAb. We first verified that the point mutation we introduced in the Fc portion of this mAb does not affect the recirculation and half-life of the mAb in vivo. The neonatal Fc receptor (FcRn) extends the half-life of IgG by reducing lysosomal degradation in endothelial cells. We thus decided to use hFcγR^{KI} hFcRn^{KI} mice (unpublished) to assess the half-life of the anti-IgE mAbs in vivo, since these mice recapitulate binding of human IgG to FcγRs and FcRn-mediated recycling of IgG. We injected 100 μg of WT or Fc-engineered ‘NA’ anti-IgE mAbs intraperitoneally (i.p.), and assessed levels of the mAbs in sera collected at different time-points. Our data demonstrate that the in vivo half-life of the WT and Fc-engineered ‘NA’ anti-IgE mAbs are not significantly different (FIG. 5). Therefore, the ‘NA’ mutation reduces binding to activating FcγRs and complement without affecting recirculation of the anti-IgE mAb in vivo.

### Example 7

Fc-Engineered Anti-IgE mAbs are as Potent as Omalizumab at Blocking IgE

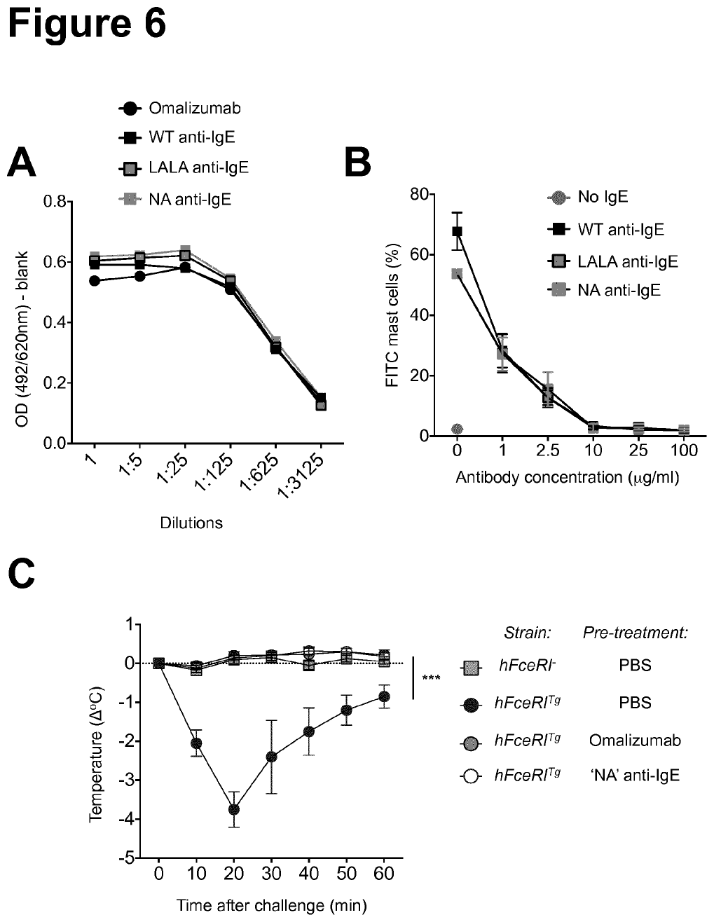

We then compared the efficiency of Omalizumab and the Fc-engineered anti-IgE mAbs at blocking IgE and IgE-mediated reactions. We first used ELISA to demonstrate that Omalizumab and the WT or Fc-engineered anti-IgE mAbs recognize human IgE with the same affinity in vitro (FIG. 6A). We then showed that the WT and Fc-engineered anti-IgE mAbs can equally block binding of human IgE and subsequent IgE-mediated degranulation in human mast cells in vitro (FIG. 6B). Finally, we showed that pre-treatment of hFcγRI^{Tg} mice (which express the human IgE high-affinity receptor FcγRI and therefore respond to human IgE) with either Omalizumab or the Fc-engineered ‘NA’ anti-IgE mAb can block human IgE-mediated passive systemic anaphylaxis (PSA) (FIG. 6C). Taken together, our results demonstrate that the Fc-engineered anti-IgE mAbs are equally potent as Omalizumab at blocking IgE-mediated allergic reactions.

### Example 8

Fc-Engineered Anti-IgE mAbs do not Induce Anaphylaxis in Humanized hFcγRKI Mice

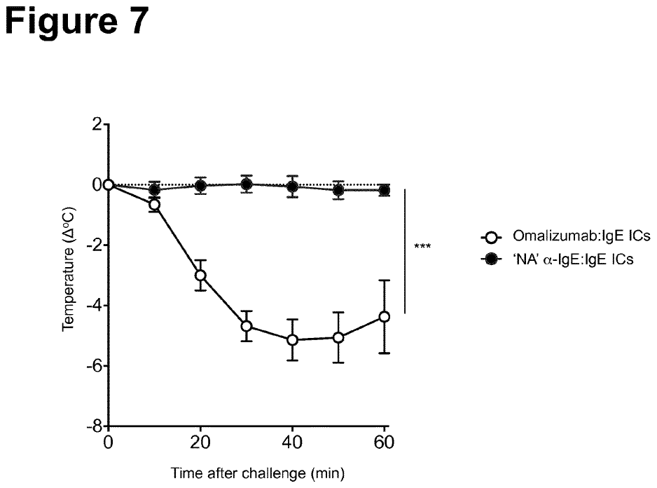

Finally, we compared the ability of ICs made of IgE and Omalizumab or IgE and the Fc-engineered ‘NA’ anti-IgE mAb to induce anaphylaxis in hFcγR^{KI} mice. As expected, we observed anaphylaxis in mice injected i.v. with ICs preformed with Omalizumab (FIG. 7). By great contrast, no sign of anaphylaxis was observed in mice injected with ICs preformed with the ‘NA’ anti-IgE mAb (FIG. 7).

### REFERENCES

Throughout this application, various references describe the state of the art to which this invention pertains. The disclosures of these references are hereby incorporated by reference into the present disclosure.
Edelman G M, Cunningham B A, Gall W E, Gottlieb P D, Rutishauser U, Waxdal M J. The covalent structure of an entire gammaG immunoglobulin molecule. Proc Natl Acad Sci USA. 1969 May; 63(1):78-85.

1. A method of treating an IgE-mediated disease in a subject in need thereof, comprising administering to the subject a therapeutically effective amount of a recombinant anti-IgE antibody,
wherein an immunoglobulin light chain protein has the amino acid sequence of SEQ ID NO: 6, and
wherein an immunoglobulin heavy chain protein has the amino acid sequence of SEQ ID NO:3 having at least one mutation in the Fc portion selected from i) a leucine (L) to alanine (A) substitution at the amino acid position corresponding to amino acid 234 of SEQ ID NO: 3 and a leucine (L) to alanine (A) substitution at the amino acid position corresponding to amino acid 235 of SEQ ID NO: 3 or ii) an asparagine (N) to alanine (A) substitution at the amino acid position corresponding to amino acid 297 of SEQ ID NO: 3.

2. The method of claim 1, wherein the IgE-mediated disease is selected from asthma and chronic idiopathic urticaria.

3. The method of claim 1, wherein the amino acid sequence of the immunoglobulin heavy chain protein is SEQ ID NO: 4.

4. The method of claim 1, wherein the amino acid sequence of the immunoglobulin heavy chain protein is SEQ ID NO: 5.

5. The method of claim 1, wherein the recombinant anti-IgE antibody is enabled to inhibit an allergic reaction without inducing anaphylaxis.

6. The method of claim 1, wherein the subject has a history of anaphylaxis.

7. A method of treating an IgE-mediated disease in a subject in need thereof, comprising administering to the subject a therapeutically effective amount of a recombinant anti-IgE antibody,
wherein an immunoglobulin light chain protein has complementarity-determining regions of the amino acid sequence of SEQ ID NO: 6, and
wherein an immunoglobulin heavy chain protein has complementarity-determining regions of the amino acid sequence of SEQ ID NO:3 and has at least one mutation in the Fc portion selected from i) a leucine (L) to alanine (A) substitution at the amino acid position corresponding to amino acid 234 of SEQ ID NO: 3 and a leucine (L) to alanine (A) substitution at the amino acid position corresponding to amino acid 235 of SEQ ID NO: 3 or ii) an asparagine (N) to alanine (A) substitution at the amino acid position corresponding to amino acid 297 of SEQ ID NO: 3.

8. The method of claim 7, wherein the IgE-mediated disease is selected from asthma and chronic idiopathic urticaria.

9. The method of claim 7, wherein the amino acid sequence of the immunoglobulin heavy chain protein is SEQ ID NO: 4.

10. The method of claim 7, wherein the amino acid sequence of the immunoglobulin heavy chain protein is SEQ ID NO: 5.

11. The method of claim 7, wherein the recombinant anti-IgE antibody is enabled to inhibit an allergic reaction without inducing anaphylaxis.

12. The method of claim 7, wherein the subject has a history of anaphylaxis.

13. A method of inhibiting anaphylaxis in a subject in need thereof, wherein the subject has an IgE-mediated disease, comprising administering to the subject a therapeutically effective amount of a recombinant anti-IgE antibody,
wherein an immunoglobulin light chain protein has complementarity-determining regions of the amino acid sequence of SEQ ID NO: 6, and
wherein an immunoglobulin heavy chain protein has complementarity-determining regions of the amino acid sequence of SEQ ID NO:3 and has at least one mutation in the Fc portion selected from i) a leucine (L) to alanine (A) substitution at the amino acid position corresponding to amino acid 234 of SEQ ID NO: 3 and a leucine (L) to alanine (A) substitution at the amino acid position corresponding to amino acid 235 of SEQ ID NO: 3 or ii) an asparagine (N) to alanine (A) substitution at the amino acid position corresponding to amino acid 297 of SEQ ID NO: 3.

14. The method of claim 13, wherein the IgE-mediated disease is selected from asthma and chronic idiopathic urticaria.

15. The method of claim 13, wherein the amino acid sequence of the immunoglobulin heavy chain protein is SEQ ID NO: 4.

16. The method of claim 13, wherein the amino acid sequence of the immunoglobulin heavy chain protein is SEQ ID NO: 5.

17. The method of claim 13, wherein the subject has a history of anaphylaxis.
